SEQUENCE LISTING (2 sequences)

SEQ ID NO: 1 - DNA, 465 bp, Homo sapiens
atggctttgggagttccaatatcagtctatcttttattcaacgcaatgacagcactgaccgaagaggcagccgtgactgtaacacctccaatcacagcccagcaaggtaactggacagttaacaaaacagaagctgacaacatagaaggacccatagccttgaagttctcacacctttgcctggaagatcataacagttactgcatcaacggtgcttgtgcattccaccatgagctagagaaagccatctgcaggtgttttactggttatactggagaaaggtgtgagcacttgactttaacttcatatgctgtggattcttatgaaaaatacattgcaattgggattggtgttggattactattaagtggttttcttgttattttttactgctatataagaaagaggtgtctaaaattgaaatcgccttacaatgtctgttctggagaaagacgaccactgtga

SEQ ID NO: 2 - protein, 154 aa, Homo sapiens
MALGVPISVYLLFNAMTALTEEAAVTVTPPITAQQGNWTVNKTEADNIEGPIALKFSHLCLEDHNSYCINGACAFHHELEKAICRCFTGYTGERCEHLTLTSYAVDSYEKYIAIGIGVGLLLSGFLVIFYCYIRKRCLKLKSPYNVCSGERRPL

(19) United States
(12) Patent Application Publication
(10) Pub. No.: US 2022/0257632 A1
(43) Pub. Date: Aug. 18, 2022

(54) MICROLUMENAL TARGETING OF CANCER CELLS

(71) Applicant: Fred Hutchinson Cancer Research Center, Seattle, WA (US)

(72) Inventors: Kevin J. Cheung, Seattle, WA (US); Emma D. Wrenn, Seattle, WA (US)

(73) Assignee: Fred Hutchinson Cancer Research Center, Seattle, WA (US)

(21) Appl. No.: 17/619,403

(22) PCT Filed: Jun. 19, 2020
(86) PCT No.: PCT/US2020/038732

§ 371 Date: Dec. 15, 2021

Related U.S. Application Data
(60) Provisional application No. 62/864,363, filed on Jun. 20, 2019.

(51) Int. Cl. A61K 31/7088; A61P 35/04; C07K 16/22; A61K 31/198; A61K 45/06; G01N 33/574; A61K 38/21

(52) U.S. Cl. CPC A61K 31/7088; A61P 35/04; C07K 16/22; A61K 31/198; A61K 2039/505; G01N 33/57488; A61K 38/217; C07K 2317/76; A61K 45/06

(57) ABSTRACT

The disclosure provides methods and compositions for microlumen targeting in the treatment of cancers characterized by solid tumors or cell clusters, such as circulating tumor cell (CTC) clusters and disseminated tumor cell (DTC) clusters. The methods and compositions specifically target epigen in microlumenal space between two or more cancer cells and reduce the expression, functionality and/or concentration of epigen within the microlumenal space. The methods and compositions can be part of methods of treating cancer in a subject wherein the cancer is characterized by a solid tumor, a circulating tumor cell (CTC) cluster, and/or a disseminated tumor cell (DTC) cluster. The reduced levels of functional epigen in the microlumen result in reduced incidence of metastasis and increased susceptibility of the tumor cells to additional therapeutic interventions.

### CROSS-REFERENCE TO RELATED APPLICATION

This application claims the benefit of U.S. Provisional Application No. 62/864,363, filed Jun. 20, 2019, which is incorporated herein by reference in its entirety.

### STATEMENT OF GOVERNMENT LICENSE RIGHTS

This invention was made with Government support under W81XWH-18-1-0098 awarded by the Department of Defense. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 72094_Sequence_final_2020-06-18.txt. The text file is 2.32 KB; was created on Jun. 18, 2020; and is being submitted via EFS-Web with the filing of the specification.

### BACKGROUND

Over 50 years ago, Fidler and Liotta used xenotransplantation assays to demonstrate that tumor cell clusters have metastatic potential superior to that of single tumor cells. More recent studies in mouse models of breast cancer have shown that between 50 and 97% of metastases arise from tumor cell clusters, illustrating the outsized potential of these tumor cell clusters to give rise to spontaneous metastases. In addition, circulating tumor cell clusters have been identified in the blood of patients in at least ten different tumor types and have been associated with poor prognosis and therapy resistance. Yet despite these robust observations across models, patients, and tumor types, the reasons why tumor cell clusters are highly metastatic have remained unclear.

A need remains for an understanding of cluster-associated metastasis and compositions and methods for preventing the same.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of inhibiting growth or metastasis of a tumor or cluster of tumor cells. The method comprises reducing the concentration of functional epigen in a microlumenal space between two or more cells of the tumor or tumor cell cluster.

In another aspect, the disclosure provides a method of treating a cancer in a subject in need thereof, wherein the cancer is characterized by a solid tumor, a circulating tumor cell (CTC) cluster, and/or a disseminated tumor cell (DTC) cluster. The method comprises administering to the subject an effective amount of an agent that reduces the functional concentration of epigen in an extracellular microlumenal space between two or more cells of the solid tumor, CTC cluster, or DTC cluster.

In another aspect, the disclosure provides a method of determining whether tumor cells are cluster dependent. The method comprises obtaining a cluster of tumor cells; and detecting spatial distribution of a low-affinity EGFR ligand within the cluster of tumor cells. Localization of the low-affinity EGFR ligand within a microlumenal space between two or more tumor cells of the cluster indicates the cluster dependency of the tumor cells.

In another aspect, the disclosure provides a method of treatment of breast cancer, such as a metastatic and/or triple-negative breast cancer, comprising administering a therapeutically effective amount of interferon gamma to a subject in need thereof. The method can include a further step of administering an antiproliferative agent, such as a tyrosine kinase inhibitor, an EGFR inhibitor, and other agents, to the subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1D show RNA-seq analysis of tumor cell aggregation and identifies epigen as a cluster-upregulated gene supporting metastatic outgrowth. MMTV-PyMT tumor organoids dissociated to single cells spontaneously aggregate into tumor cell clusters in non-adherent conditions; equal numbers of dissociated single tumor cells were aggregated for varying times then subjected to (1) tail vein metastasis assays and (2) RNA-seq to identify genes induced by the clustering process (FIG. 1A). MMTV-PyMT mTomato+ tumor organoids were enzymatically dissociated to single cells and aggregated for 0, 6, and 24 hrs. As an additional control, at 24 hrs, some clusters were re-dissociated back to single cells. At each time point 200,000 cells were injected by tail vein into NSG mice. # of mTomato+ metastases. n (# mice)=19 (0 hr), 9 (6 hr), 14 (24 hr), 10 (re-dissociated). P-values=Mann-Whitney tests (FIG. 1B). The top ten cluster-induced genes (by t-statistic) compared between 0 and 6 hrs. n=3 biological replicates (FIG. 1C). Total GFP+ lung metastatic burden per mouse, relative to mean Ctrl-kd metastatic burden for each injection replicate. n (# of mice): Ctrl-kd=31, Egfr-kd=8, Areg-kd=8, Epgn-kd=12, Plaur-kd=8. One-way ANOVA p<0.0001. P-values=Dunnett's Test (FIG. 1D).

FIGS. 2A-2D show that epigen suppression switches tumor cell clusters from a proliferative state to a migratory state. Gene ontology analysis of genes significantly upregulated or downregulated in Epgn-kd clusters compared to Ctrl-kd clusters after 24 hrs of aggregation (Metascape, FDR≤0.05) (FIG. 2A). Time-lapse images of Ctrl-kd and Epgn-kd MMTV-PyMT clusters in 3D basement membrane-rich gels. Fold change in cluster area and the centroid position of each cluster (relative to landmarks in the 3D gel) were measured every 10 hrs. Right, summary of cluster size and location over 120 hrs (FIG. 2B). Cumulative migration distance, normalized to initial cluster cell number, for Ctrl-kd and Epgn-kd clusters over 120 hrs in 3D basement membrane-rich gels. n (# of clusters): Ctrl-kd=106, Epgn-kd=102. n (# of biological replicates)=4. P-values=unpaired t-tests (FIG. 2C). Area measurements, relative to starting cluster area, of Ctrl-kd and Epgn-kd clusters cultured in 3D basement membrane-rich gels. Mean with 95% CI. n (# of clusters): Ctrl-kd=106, Epgn-kd=102. n (# of biological replicates)=4. P-values=unpaired t-tests (FIG. 2D).

FIGS. 3A-3F show that epigen acts as a collective signaling factor shared non-cell-autonomously within clusters. FIG. 3A shows possible mechanisms by which epigen is a signal for cluster outgrowth. FIG. 3B shows time-lapse fold outgrowth measurements of (B). n=71 non-transduced clusters cultured alone, 22 non-transduced clusters in co-culture, 51 Epgn-kd clusters cultured alone, 44 Epgn-kd clusters in co-culture. n=2 biological replicates. P-values=Mann-Whitney test. FIG. 3C shows immunofluorescence of epigen in a MMTV-PyMT cluster ex vivo; the graph represents quantification of epigen signal. n=3 biological replicates, n=63 clusters. P-values=Mann-Whitney tests. FIG. 3D shows immunofluorescence of epigen in Ctrl-kd and Epgn-kd clusters; quantification of mean epigen intensity along intracellular, cell-matrix, or cell-cell regions. n=2 biological replicates, 30 clusters per condition. P-values=Mann-Whitney test. FIG. 3D is time lapse images of Epgn-kd-GFP and non-transduced cells aggregated in different ratios to form pure or mosaic non-transduced/Epgn-kd clusters. All cells are mTomato+, Epgn-kd cells are GFP+. FIGS. 3E and 3H demonstrated rescue of Epgn-kd cell growth by neighboring Epgn+ cells thus supporting model 4. Outgrowth of Epgn-kd-GFP or Egfr-kd-GFP cells when mixed with different proportions of non-transduced cells. (FIG. 3E). Outgrowth was normalized to 100% knockdown cell clusters. Epgn-kd n=3 biological replicates (36,022 cells). Egfr-kd n=4 biological replicates (19,911 cells). Median and 95% CI. P-values=unpaired t-tests vs. pure (100%) knockdown clusters

FIGS. 4A-4E show that epigen is stored and concentrated within largely impermeable intercellular microlumina. FIG. 4A shows TEM images of MMTV-PyMT clusters. MP=microvilli-like protrusions. J=cell-cell junction.

FIG. 4B shows possible models of intercellular epigen signaling either as a membrane-restricted juxtacrine ligand or a freely diffusing paracrine ligand. FIG. 4C is a quantification of epigen immunogold signal; n=268 gold particles. To determine if microlumenal junctions were permeable, clusters were incubated with cell-impermeable sulfo-NHS-biotin. 1 mM EGTA was used as a positive control to disrupt cell-cell junctions. Biotin leak was quantified by measuring streptavidin-FITC fluorescence signal within clusters. n=3 biological replicates. n=75 clusters per condition. P-values=Mann-Whitney tests (FIG. 4D) Immunofluorescence of epigen in clusters with and without 1 mM EGTA treatment to disrupt cell-cell junctions was measured. FIG. 4E shows quantification of median epigen intensity within clusters. n=3 biological replicates, 29 untreated clusters, 30 EGTA treated clusters. P-value=Mann-Whitney test.

FIGS. 5A-5H show that epigen suppression reduces both primary and metastatic tumor outgrowth. FIG. 5A shows quantification of epigen expression in single tumor cells and rounded/non-protrusive clusters in adjacent tumor stroma. n=27 single cells, 33 clusters. n=2 tumors. P-values=Mann-Whitney tests. FIG. 5B shows quantification of relative epigen IF intensity in adjacent non-protrusive and protrusive clusters. n=3 tumors. n=29 non-protrusive, 31 protrusive clusters. P-value=Mann-Whitney test. FIG. 5C shows intensity of epigen immunofluorescence in locally disseminated clusters adjacent to brain metastases. n=3 brains. n=12 non-protrusive, 9 protrusive clusters. P-value=Mann-Whitney test. FIG. 5D shows quantification FITC-streptavidin intensity in tumor nests (MMTV-PyMT primary tumors treated with sulfo-NHS-biotin) compared to adjacent stroma. n=3 tumors, 50 tumor areas (“t”), 50 stromal (“s”) areas. P-value=Mann-Whitney test. FIG. 5E is a quantification of biotin leak (internal intensity/external intensity) for non-protrusive vs. protrusive clusters. n=3 tumors. n=9 non-protrusive, 12 protrusive clusters. P-value=Mann-Whitney test. FIG. 5F is a FITC-streptavidin intensity in lung metastasis nests vs. adjacent stroma. n=3 mice, 50 lung metastasis areas, 32 stromal areas. P-value=Mann-Whitney test. FIG. 5G is a graph of estimated tumor volume of primary tumors formed after orthotopic transplant of Ctrl-kd or Epgn-kd clusters. n=10 Ctrl-kd mice, 9 Epgn-kd mice. P-value=Mann-Whitney test. FIG. 5H shows that lung metastases formed 6 weeks after orthotopic transplant of Ctrl-kd or Epgn-kd clusters (quantification of visible GFP+ lung metastases using stereomicroscope. n=10 Ctrl-kd mice, 9 Epgn-kd mice. P-value=Mann-Whitney test).

FIGS. 6A-6E show that high epigen expression and microlumina formation are associated with the basal-like 2 subgroup of triple-negative breast cancer. FIG. 6A shows clinical progression of a patient with metastatic ER+/PR+/HER2-breast cancer from whom malignant ascites were collected. Treatment course: (1) vinorelbine, (2) cytoxan/methotrexate, (3) drug holiday, (4) cytoxan/methotrexate, (5) irinotecan, (6) low dose doxorubicin, (7) hospice. LDH=serum lactate dehydrogenase. CA15.3=cancer antigen 15-3. FIG. 6B shows epigen mRNA expression in human cell lines classified by different triple-negative breast cancer sub-groups. P-values=Kolmogorov-Smirnov test. FIG. 6C shows test of cluster permeability using sulfo-NHS-biotin: quantification of biotin leak (internal biotin signal/membrane biotin signal) in clusters. n (# clusters/# biological replicates): HCC70=25/2, CAL851=30/3, HDQP1=30/3, MDA-MB-231=17/2, MDA-MB-436=30/3, BT549=30/3. FIG. 6D shows gene sets (Metascape, FDR≤0.01) enriched in BL2 (HCC70, CAL851, HDQP1) vs. M-like (MDA-MB-231, MDA-MB-436, and BT549) cell lines (CCLE). FIG. 6E shows RNA expression of individual genes (CCLE) in BL2 (HCC70, CAL851, HDQP1) vs. M-like (MDA-MB-231, MDA-MB-436, and BT549) cell lines.

FIGS. 7A-7J show that HCC70 outgrowth depends on epigen expression and is exquisitely sensitive to IFNγ which induces microlumen permeability. FIG. 7A is a qPCR measurement of EPGN expression in HCC70 clusters. n=3 biological replicates. P-values=unpaired t-tests. FIG. 7B is shows cell counts after 6 days of culture, plated at equal starting cell densities, of HCC70 clusters. n=3 biological replicates. P-values=unpaired t-tests. FIG. 7C is a quantification of individual lung metastasis areas (lungs three weeks after tail vein injection of 200,000 Ctrl-kd or Epgn-kd HCC70 clustered cells). n (# of metastases): Ctrl-kd=299, Epgn-kd=87. n (# of mice): 7 per condition. Box plot=5-95%. P-value=Mann-Whitney test. FIG. 7D shows viable cell quantification after 6 days of IFNγ treatment. Line=non-linear regression. Band=95% CI. n=3 biological replicates. P-value=extra sum-of-squares F test. FIG. 7E shows pHH3 immunofluorescence to mark mitotic cells in HCC70 clusters after 6 days with or without 3 ng/mL IFNγ treatment (quantification of % pHH3+ nuclei in clusters from each condition). n=3 biological replicates. n=40 untreated clusters, 34 IFNγ treated clusters. P-value=Mann-Whitney test. FIG. 7F is a quantification of internal FITC-streptavidin intensity biotin leak-in assay of HCC70 clusters after 6 days of treatment with or without 3 ng/mL IFNγ). n=60 untreated clusters, 70 IFNγ treated clusters. n=3 biological replicates. P-value=Mann-Whitney test. FIG. 7G is a TEM of HCC70 clusters after 6 days with and without IFNγ treatment (3 ng/mL). MP=microvilli-like protrusions. J=cell-cell junctions. FIG. 7H is a qPCR of (left) RAB25 and (right) CLDN7 in HCC70 clusters after 0, 3, and 6 days with and without 3 ng/mL IFNγ treatment. n=3 biological replicates. P-value=unpaired t-test. FIGS. 7I and 7J: HCC70 single cells were aggregated for 24 hrs then treated with or without 2 ng/mL IFNγ treatment. After 6 days of treatment, clusters from each condition were injected by tail vein into NSG mice, with 200,000 viable clustered cells injected per animal. 3 weeks later, lung metastases were scored by staining for the human-specific marker CD298. Sizes of individual HCC70 metastases at 3 weeks after tail vein injection. n=8 mice/condition, 217 untreated metastases, 79 IFNγ pre-treated metastases. Box plot=5-95%. P-value=Mann-Whitney test (7I). Number of hCD298+ lung metastases per mouse 3 weeks after HCC70 cluster tail vein injection with or without IFNγ (2 ng/mL) pre-treatment. n=8 mice/condition. P-value=Mann-Whitney test (7J).

FIG. 8 is a quantification of double positive (GFP+ and mTomato+) and shRNA escape (mTomato+but GFP−) areas in Ctrl-kd or Epgn-kd lung metastases 3 weeks after tail vein injection. n=3 mice per condition. P-values=unpaired t-tests.

FIG. 9 illustrates the discovery that microlumina are hubs for epigen collective signaling during multicellular metastatic outgrowth. Tumor cell clusters generate sealed, intercellular microlumina. During phases of outgrowth, these microlumina are largely impermeable and contain high concentrations of the growth factor epigen. Intercellular, collective signaling by epigen within cluster microlumina promotes primary and metastatic tumor outgrowth. In contrast, low epigen expression is found in highly protrusive locally disseminated clusters, which have increased junctional permeability. High epigen expression and microlumenal impermeability were found in basal-like 2 human breast cancer cells, whereas mesenchymal-like cells were highly permeable and lowly expressed epigen. Suppression of epigen, or targeting of microlumenal junctions, in basal-like 2 breast cancer cells disrupt collective metastasis.

### DETAILED DESCRIPTION

Tumor cell clusters form metastases more efficiently than single cells, but the molecular mechanisms explaining this phenomenon have remained obscure. As described herein, the inventors have demonstrated that breast tumor cell clusters form microlumen-intercellular cavities—with high concentrations of the growth factor and EGFR ligand, epigen. Epigen or EGFR knockdown strongly suppresses metastatic outgrowth of tumor cell clusters. Epigen and active EGFR colocalize in intercellular spaces in primary tumors and metastases. Mechanistically, epigen acts as a short-range growth signal sequestered in microlumen and shared between clustered cells. Disrupting cell-cell junctions blocks epigen accumulation and suppresses outgrowth. Ultimately, the inventors' work reveals that tumor cell clusters utilize tissue architecture to restrict epigen diffusion and focus growth signaling and targeting microlumenal signaling can limit metastasis.

In accordance with the foregoing, in one aspect, the disclosure provides a method of inhibiting growth or metastasis of a tumor or cluster of tumor cells. The method comprises reducing the concentration of functional epigen in a microlumenal space between two or more cells of the tumor or cluster of tumor cells.

The term “tumor” as used herein refers broadly to an aggregation of two or more transformed cells that aberrant gene expression or phenotype relative to the cell or tissue type from which the cells were transformed. At the least, the transformed cells lose one or more functional characteristics that define the source tissue or cells from which they transform. A tumor can be benign, pre-malignant, or malignant. Pre-malignant tumor cells have qualities that indicate they are developing into malignant cells. Malignant tumor cells encompass cancer cells and are characterized by increased growth and division rates, and can invade other areas of the body, including other tissues (i.e., metastasize). Malignant tumor cells include, e.g., carcinoma cells, sarcoma cells, germ cell tumors, or blastoma cells. The term “cluster of tumor cells” refers to an aggregation of transformed cells that adhere to each other through, e.g., tight junctions, that maintain cell-to-cell contact. The clusters can be circulating tumor cells (CTCs), which are released from solid tumors into the surrounding environment. CTCs can migrate or circulate around the body and invade into distinct tissues and locations from the source tumor. If the CTCs survive, they can become established in a new location giving rise to disseminated tumor cells (DTCs) and overt metastatic disease. Accordingly, production and survival of CTCs to establish DTCs provide a primary mechanism for metastasis. Clusters of CTCs and DTCs are encompassed by this disclosure.

In some embodiments, the tumor is a breast cancer tumor. In some embodiments, the breast cancer is a metastatic breast cancer. In some embodiments, the breast cancer is a triple-negative breast cancer, such as BL2 triple-negative breast cancer. In some embodiments, the cluster of tumor cells is derived from (or produced by) a breast cancer tumor. In this regard, the tumor originated from cells that were transformed from cells in breast tissue.

The method is directed to inhibiting of growth or metastasis of the tumor or cluster of tumor cells. The term “inhibition of growth” encompasses a slowing of growth or cell division rates. The term “inhibition of metastasis” encompasses a reduction in rate or risk of metastasis in vivo. This includes a reduction in production of CTC clusters from a tumor, a reduction in the ability of CTC clusters to survive in circulation, and/or a reduction in the ability of CTCs to establish a DTC cluster in a location apart from the source tumor. Additionally, in some embodiments, the method also encompasses increasing vulnerability of the tumor cells, including CTC and DTC clusters to other cancer therapies, including EGFR inhibition. In in vivo embodiments, the method can be performed on any subject with metastatic or premetastatic cancer. The subject can be, e.g., a human or non-human mammal, such as another primate, horse, dog, mouse, rat, guinea pig, rabbit, and the like.

Epigen, also known as epithelial mitogen, is a protein encoded the EPGN gene (GenBank gene ID 255324). A representative protein sequence for Epigen is provided in Uniprot Accession No. Q6UW88, incorporated herein by reference in its entirety, and is also set forth herein as SEQ ID NO:2. Epigen is part of the epidermal growth factor family and is known to function as a ligand for the epidermal growth factor receptor (EGFR).

The term “reducing the concentration of functional epigen in a microlumenal space” encompasses reducing the expression of epigen in the microlumenal space, interfering with the functionality of intact (e.g., wild-type epigen) in the microlumenal space, and reducing the concentration of intact (e.g., functional or wild-type) epigen in the microlumenal space.

The term “microlumenal space” refers to space that exists between two or more cells (e.g., tumor cells) that otherwise maintain cell-to-cell contacts (e.g., via junctional adhesions). As described below, the microlumenal spaces appear as enclosed hollow pockets or gaps between neighboring cells in tumor cell clusters. The microlumenal spaces are observable after about 6 hours of cluster formation. While the microlumenal pockets present in a variety of shapes and configurations, many have at least one exemplary dimension (i.e., length and/or width) on a scale from about 10 nm (near tight junctions or between extending vili) to about 1 min or even more. Regarding the irregular dimensions, the intact microlumenal pockets are typically enclosed spaces that are effectively sealed off from other distinct extracellular spaces that may surround the cell cluster by tight junctions maintained between the neighboring cells. The microlumenal space can be defined by (i.e., disposed between) the cells walls of a plurality of neighboring cells in a cluster, such as 2, 3, 4, 5, 6, 7, 8, 9, 10, or more neighboring cells.

In one embodiment, the method comprises reducing expression of epigen in one or more solid tumor cancer cells defining the boundary of the microlumenal space. Without being limited to a particular theory, reduction of cell production of epigen by the tumor or cluster cell will reduce the source of epigen that would otherwise be delivered to the microlumenal space between the cells of the tumor or tumor cluster. Expression of epigen in the solid tumor cancer cells can be reduced by inducing RNA interference of the translation of epigen from the encoding mRNA transcript. RNA interference (“RNAi”) is a biological process in which RNA molecules inhibit gene expression or translation, by neutralizing targeted mRNA molecules. Inducing RNA can include administering to the microlumenal space RNA constructs, e.g., microRNA (miRNA) or small interfering RNA (siRNA), that form double stranded RNA associations with the mRNA that encodes epigen. The formation of such double stranded associations recruits endogenous enzyme complexes that degrade the mRNA, thus removing their availability for translation. This technique can be implemented to target any mRNA transcript encoding an epigen protein with, for example, a sequence set forth in SEQ ID NO:2. An exemplary nucleic acid sequence representing the epigen transcript is provided in SEQ ID NO:1. A person of ordinary skill in the art can readily design various embodiments of miRNA and/or siRNA operative to induce RNA interference and, thus, reduce the expression of functional epigen in the one or more solid tumor cancer cells defining the boundary of the microlumenal space.

In other embodiments, the step of reducing the concentration of functional epigen comprises administering to the microlumenal space an affinity reagent that selectively binds epigen. In other embodiments, the step of reducing the concentration of functional epigen comprises administering to the microlumenal space an affinity reagent competes with epigen for binding to a cell surface receptor. In either embodiment, the affinity reagent disrupts the ability of epigen to bind with its cognate receptor, e.g., EGFR, thus preventing its typical function within the tumor or tumor cell cluster.

An affinity reagent is a molecule that exhibits specific binding properties to a target molecule (e.g., epigen or a cognate receptor for epigen such as EGFR). The term “specifically bind” or variations thereof refer to the ability of the affinity reagent (e.g., of an antibody, or fragment or derivative thereof) to bind to the antigen of interest (e.g., epigen or EGFR), without significant binding to other molecules, under standard conditions known in the art. The binding domain can bind to other peptides, polypeptides, or proteins, but with lower affinity as determined by, e.g., immunoassays, BIAcore, or other assays known in the art. However, the affinity reagent preferably does not substantially cross-react with other antigens.

In some embodiments, the affinity reagent is an antibody, or antibody fragment or derivative. The term “antibody” is used herein in the broadest sense and encompasses various antibody structures derived from any antibody-producing mammal (e.g., mouse, rat, rabbit, and primate including human), and which specifically bind to an antigen of interest (e.g., epigen or EGFR). An antibody fragment specifically refers to an intact portion or subdomain of a source antibody that still retains antigen-biding capability. An antibody derivative refers to a molecule that incorporates one or more antibodies or antibody fragments. Typically, in a derivative there is at least some additional modification in the structure of the antibody or fragment thereof, or in the presentation or configuration of the antibody or fragment thereof. Exemplary antibodies of the disclosure include polyclonal, monoclonal and recombinant antibodies. Exemplary antibodies or antibody derivatives of the disclosure also include multispecific antibodies (e.g., bispecific antibodies); humanized antibodies; murine antibodies; chimeric, mouse-human, mouse-primate, primate-human monoclonal antibodies; and anti-idiotype antibodies.

As indicated, an antibody fragment is a portion or subdomain derived from or related to a full-length antibody, preferably including the complementarity-determining regions (CDRs), antigen binding regions, or variable regions thereof, and antibody derivatives refer to further structural modification or combinations in the resulting molecule. Illustrative examples of antibody fragments or derivatives encompassed by the present disclosure include Fab, Fab′, F(ab)₂, F(ab′)₂ and Fv fragments, diabodies, single-chain antibody molecules, V_{H}H fragments, V_{NAR} fragments, multispecific antibodies formed from antibody fragments, nanobodies and the like. For example, an exemplary single chain antibody derivative encompassed by the disclosure is a “single-chain Fv” or “scFv” antibody fragment, which comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Another exemplary single-chain antibody encompassed by the disclosure is a single-chain Fab fragment (scFab).

As indicated, antibodies can be further modified to created derivatives that suit various uses. For example, a “chimeric antibody” is a recombinant protein that contains domains from different sources. For example, the variable domains and complementarity-determining regions (CDRs) can be derived from a non-human species (e.g., rodent) antibody, while the remainder of the antibody molecule is derived from a human antibody. A “humanized antibody” is a chimeric antibody that comprises a minimal sequence that conforms to specific complementarity-determining regions derived from non-human immunoglobulin that is transplanted into a human antibody framework. Humanized antibodies are typically recombinant proteins in which only the antibody complementarity-determining regions (CDRs) are of non-human origin. Any of these antibodies, or fragments or derivatives thereof, are encompassed by the disclosure.

Antibody fragments and derivatives that recognize specific epitopes can be generated by any technique known to those of skill in the art. For example, Fab and F(ab′)₂ fragments of the disclosure can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies, or fragments or derivatives thereof, of the present disclosure can also be generated using various phage display methods known in the art. Finally, the antibodies, or fragments or derivatives thereof, can be produced recombinantly according to known techniques.

It will be apparent to the skilled practitioner that the affinity reagent can comprise antigen binding molecules other than antibody-based domain, such as peptidobodies, antigen-binding scaffolds (e.g., DARPins, HEAT repeat proteins, ARM repeat proteins, tetratricopeptide repeat proteins, and other scaffolds based on naturally occurring repeat proteins, etc. [see. e.g., Boersma and Pluckthun, Curr. Opin. Biotechnol. 22:849-857, 2011, and references cited therein, incorporated herein by reference]), which include a functional binding domain or antigen-binding fragment thereof.

In yet other embodiments, the step of reducing the concentration of functional epigen comprises administering an effective amount of an agent that disrupts cell to cell contact of two or more cells defining the boundary of the microlumenal space. By disrupting the integrity of the cell wall contacts that define the boundaries of the microlumenal space, the contents of the microlumenal space can escape or be diluted with the incursion of additional fluid. Accordingly, in some embodiments, the agent disrupts cell-to-cell contacts of the cells of the tumor or cluster of tumor cells. The agent can disrupt, for example, tight junctions. In some embodiments, the agent is a calcium chelator. Exemplary, non-limiting examples of calcium chelator that can disrupt cell-to-cell contacts ethylene glycol-bis(β-aminoethyl ether)-N,N,N′,N′-tetraacetic acid (EGTA), ethylenediaminetetraacetic acid (EDTA), and the like.

In another aspect, the disclosure provides a method of treating a cancer in a subject in need thereof. The cancer is characterized by a solid tumor, a circulating tumor cell (CTC) cluster, and/or a disseminated tumor cell (DTC) cluster. The method comprises administering to the subject an effective amount of an agent that reduces the functional concentration of epigen in an extracellular microlumenal space between two or more cells of the solid tumor, CTC cluster, or DTC cluster.

As used herein, the term “treat” refers to medical management of a disease, disorder, or condition (e.g., cancer, as described above) of a subject (e.g., a human or non-human mammal, such as another primate, horse, dog, mouse, rat, guinea pig, rabbit, and the like). Treatment can encompasses any indicia of success in the treatment or amelioration of a disease or condition (e.g., a cancer), including any parameter such as abatement, remission, diminishing of symptoms or making the disease or condition more tolerable to the subject, slowing in the rate of degeneration or decline, and/or making the degeneration less debilitating. Specifically, in the context of cancer, the term treat can encompass slowing or inhibiting the rate of cancer growth, or reducing the likelihood of recurrence, and/or preventing or reducing the risk of metastasis compared to not having the treatment. In some embodiments, the treatment encompasses resulting in some detectable degree of cancer cell death in the patient. The treatment or amelioration of symptoms can be based on objective or subjective parameters, including the results of an examination by a physician. Accordingly, the term “treating” includes the administration of the compositions of the present disclosure to alleviate, or to arrest or inhibit development of the symptoms or conditions associated with disease or condition (e.g., cancer). The term “therapeutic effect” refers to the amelioration, reduction, or elimination of the disease or condition, symptoms of the disease or condition, or side effects of the disease or condition in the subject. The term “therapeutically effective” refers to an amount of the composition that results in a therapeutic effect and can be readily determined.

In some embodiments, reducing the concentration of functional epigen comprises administering an effective amount of an agent that disrupts cell to cell contact of two or more cancer cells defining the boundary of the microlumenal space. In some embodiments, the agent disrupts calcium-dependent cell to cell contacts. In such embodiments, the agent can be a calcium chelator. Nonlimiting examples of calcium chelators encompassed by the present disclosure include ethylene glycol-bis(β-aminoethyl ether)-N,N,N′,N′-tetraacetic acid (EGTA), and the like.

In some embodiments, the method is an aspect of a combination therapy and thus further comprises administering to the subject an effective amount of another agent to treat the cancer. The anti-cancer therapeutic can be any toxin, small molecule, large molecule therapeutic that has demonstrable therapeutic effect on cancer cells in a subject. In some embodiments, the additional agent is an EGFR inhibitor. In some embodiments, the other anti-cancer agent is an immunotherapeutic agent, such as cancer-specific antibodies or functional fragments thereof, immune checkpoint inhibitors, and adoptive cell therapies, including CAR T-cell therapy. The immunotherapeutic agent can be an antibody or functional fragment thereof that substantially targets cancers. Substantially targets means that the agent primarily affects cancer cells with ideally reduced off-target action. However, it is not a requirement that the agent completely avoids off-target activity and has no detrimental side-effects. In some embodiments, the additional therapeutic agent comprises therapeutic antibodies (or fragments thereof) that target EGFR, HER2, and HER3. In some embodiments, the anti-cancer therapeutic comprises kinase inhibitors, such as tyrosine kinase inhibitors. One example of a tyrosine kinase inhibitor is afatinib. In some embodiments, the agent is a small molecule inhibitor of HER2, for example, tucatinib. In some embodiments, the additional immunotherapeutic agent comprises an immune cell modified or expanded ex vivo and expresses a receptor specific for the cancer, such as a CAR-T cell.

As described in more detail below, the inventors discovered that tumor cells can exhibit enhanced characteristics, including resiliency to some treatments and enhanced ability to establish metastasis when they maintained a cluster formation. The inventors determined that the clusters of these cells create and advantageous architecture whereby microlumenal space can maintain an aggregation of EGFR ligands, including epigen. Thus, in another aspect, the disclosure provides a method of determining whether tumor cells are cluster-dependent. The method comprises:

obtaining a cluster of tumor cells; and

detecting spatial distribution of a low-affinity EGFR ligand within the cluster of tumor cells;

wherein localization of the low-affinity EGFR ligand within a microlumenal space between two or more tumor cells of the cluster indicates the cluster dependency of the tumor cells.

The low-affinity EGFR ligand can be selected from epigen, amphiregulin, and epiregulin. Detection of the low-affinity EGFR ligand can incorporate contacting the cluster of tumor cells (e.g., the CTC cluster or DTC cluster) with an affinity reagent, such as an antibody, or fragment or derivative thereof, which specifically binds to the low-affinity EGFR ligand. The affinity reagent can be detectably labeled or can otherwise be selectively bound by a second affinity reagent that is detectably labeled. The detectable label can then be observed by, e.g., immuno-electron microscopy, or other techniques that can quantify binding of the affinity reagent to the low affinity EGFR ligand.

The tumor cells determined to be cluster-dependent can have elevated resistance to immunotherapeutic agents compared to tumor cells that are not cluster-dependent.

In some embodiments, the cluster of tumor cells is obtained from a subject with a solid tumor, wherein a determination that the tumor cells in the cluster are cluster-dependent indicates the subject has a tumor that is cluster-dependent. With such a determination, the method can further comprise administering to the subject an effective amount of an agent that disrupts cell to cell contact of two or more cells defining the boundary of microlumenal space in the tumor. As indicated above, the agent can be characterized by the ability to disrupt calcium-dependent cell to cell contact. In some embodiments, the agent is a calcium chelator, such as ethylene glycol-bis(β-aminoethyl ether)-N,N,N′,N′-tetraacetic acid (EGTA), and the like. The method can further comprise administering to the subject an effective amount of an immunotherapeutic agent for the cancer, as described above.

In another aspect, provided herein is a method of treatment of breast cancer in a subject in need thereof comprising administering a therapeutically effective amount of interferon gamma to the subject. In some embodiments, the breast cancer is a metastatic breast cancer. In some embodiments, the breast cancer is a triple-negative breast cancer. In some embodiments, the breast cancer is basal-like-2 (BL2) triple-negative breast cancer. In some embodiments, the breast cancer is not mesenchymal-like triple-negative breast cancer. In some embodiments, the breast cancer is a breast cancer characterized by increased epigen expression. In some embodiments, the breast cancer is ER+ breast cancer.

In some embodiments, the method further comprises administering an anti-cancer therapeutic agent, such as any agent used as a standard of care therapy for metastatic breast cancer. In some embodiments, administration of interferon gamma can be done in conjunction with any standard of care therapy for metastatic breast cancer. Suitable combinations include combination of interferon gamma with an agent selected from EGFR family (HER1-4)-targeted therapy (e.g. TKIs, anti-EGFR antibody, and HER2-targeted therapy), immunotherapy (e.g. immune checkpoint inhibitors, e.g. atezolizumab and pembrolizumab), ADC-conjugated antibodies (e.g. sacituzumab which is also approved), PARP inhibitors (such as olaparib), and standard-of-care chemotherapy (including microtubule targeted agents such as paclitaxel and eribulin, anthracyclines such as doxorubicin, antimetabolites such as capecitabine and gemcitabine and methotrexate, platinum agents such as cisplatin), and combinations thereof. In some embodiments, interferon gamma can be administered with endocrine-targeted therapy such as estrogen receptor antagonists (e.g. fulvestrant or a CDK4/6 inhibitor such as palbociclib, ribociclib, and abemaciclib). In some embodiments of the methods, the agent is atezolizumab, pembrolizumab, talazoparib, olaparib, lapatinib, neratinib, tucatinib, palbociclib, ribociclib, abemaciclib, sacituzumab, fulvestrant, doxorubicin, capecitabine, gemcitabine, paclitaxel, eribulin, methotrexate, cisplatin, or a combination thereof.

In some embodiments, administration of interferon gamma and the anti-cancer agent can be performed sequentially in any order or simultaneously.

Thus, in another aspect, disclosed herein is a composition comprising interferon gamma and an anti-cancer agent, such as an agent that is used as a standard of care therapy for metastatic breast cancer. The compositions can optionally include one or more pharmaceutically acceptable carrier. In some embodiments of the compositions, the agent is atezolizumab, pembrolizumab, talazoparib, olaparib, lapatinib, neratinib, tucatinib, palbociclib, ribociclib, abemaciclib, sacituzumab, fulvestrant, doxorubicin, capecitabine, gemcitabine, paclitaxel, eribulin, methotrexate, cisplatin, or a combination thereof. The gamma interferon and the anti-cancer agent can be included in separate containers, each of which can optionally comprise a pharmaceutically acceptable carrier. In some embodiments, the composition comprises a treatment regimen.

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed in this description and/or the claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:

(1) Alanine (A), Serine (S), Threonine (T),

(2) Aspartic acid (D), Glutamic acid (E),

(3) Asparagine (N), Glutamine (Q),

(4) Arginine (R), Lysine (K),

(5) Isoleucine (I), Leucine (L), Methionine (M), Valine (V), and

(6) Phenylalanine (F), Tyrosine (Y), Tryptophan (W).

Reference to sequence identity addresses the degree of similarity of two polymeric sequences, such as protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

The following examples are provided for the purpose of illustrating, not limiting, the invention.

### EXAMPLES

Collective metastasis is defined as the cohesive migration and metastasis of multicellular tumor cell clusters. Disrupting various cell adhesion genes markedly reduces cluster formation and colonization efficiency, but the downstream signals transmitted by clustering remain unknown. The inventors identified a mechanism of collective signaling used by tumor cell clusters to support metastatic colonization. In both mouse and human breast cancer models, tumor cell clusters produced the growth factor epigen and concentrate it within microlumina—intercellular cavities sealed by cell-cell junctions and lined with microvilli-like protrusions. Epigen knockdown profoundly reduced metastatic outgrowth and switches clusters from a proliferative to a collective migratory state. Tumor cell clusters from basal-like-2, but not mesenchymal-like, triple-negative breast cancer cell lines have increased epigen expression, sealed microlumina, and impaired outgrowth upon microlumenal junction disruption. Thus, microlumenal collective signaling is a therapeutic target for aggressive metastatic breast cancers.

Though metastasis is often conceived of as a single cell process, multicellular tumor cell clusters have been directly observed at tumor invasive fronts, within the systemic circulation, and colonizing distant organs. Until recently, the functional importance of clusters for metastatic seeding has remained unclear. Studies have since shown that multicellular clusters give rise to between 50-97% of metastases in different models, supporting the hypothesis for a collective origin of metastasis. Notably, circulating tumor cell clusters are associated with rapid disease progression and increased mortality in many different cancer types. Yet compared with the process of single cell metastasis, the molecular mechanisms driving dissemination and outgrowth of multicellular clusters are far less understood.

An important question is whether multicellular organization gives rise to unique cellular and molecular properties that promote metastasis and are targets for therapy. Recent studies have identified some of the features distinguishing single and clustered tumor cells: increased chromatin accessibility at the binding sites of sternness-promoting transcription factors; the ability of clusters to maintain cell-cell adhesion while squeezing through narrow vasculature, extravasating and proliferating at secondary sites; and the increased capacity of clusters to survive reactive oxygen stress, an acute challenge for disseminating cells. In several of these studies, disrupting cell adhesion genes drastically reduces cluster formation and integrity, in turn diminishing the metastatic potential of these tumor cells. Although cell-cell adhesion molecules are often abundant in normal tissues which poses a challenge, these studies advance the concept that therapeutically targeting cluster formation could robustly suppress collective metastasis.

An alternative approach is to focus on the signals arising downstream of tumor cell cluster formation. This strategy is supported by evidence that multicellularity and cell-cell adhesion regulate signaling in normal contexts to accomplish dramatic cellular rearrangements and tissue remodeling during developmental morphogenesis and wound healing. Intercellular cooperation and signaling in these systems are still incompletely understood but are known to involve synchronized shifts in the composition of cellular junctions, spatially restricted and long-range paracrine signaling, contact-mediated juxtacrine signaling, and patterning by the local microenvironment. Thus, in addition to forming adhesions, clustering of tumor cells could give rise to complex signaling mechanisms distinct from those utilized by single cells.

Further, metastasis has been conceptualized as a series of switch-like transitions that involve the collaboration of tumor cell communities. Although a number of theoretical studies have posited that cancer cells could promote metastatic transitions through cooperative signaling mechanisms, direct molecular evidence for collective signaling between tumor cells during metastasis remains scarce.

In the present disclosure, mouse and human models of breast cancer metastasis were used to identify collective signals generated by tumor cell clusters during metastatic outgrowth. The inventors showed that mouse and human tumor cell clusters harbor microlumina—intercellular signaling compartments sealed by cell-cell junctions. Within microlumina, a diffusible growth factor, epigen, which controls switching between collective migratory and proliferative modes was identified. Importantly, it was found that epigen expression and microlumen formation are enriched in a specific subtype of aggressive, triple-negative human breast cancers.

Metastatic Potential Increases During Clustering

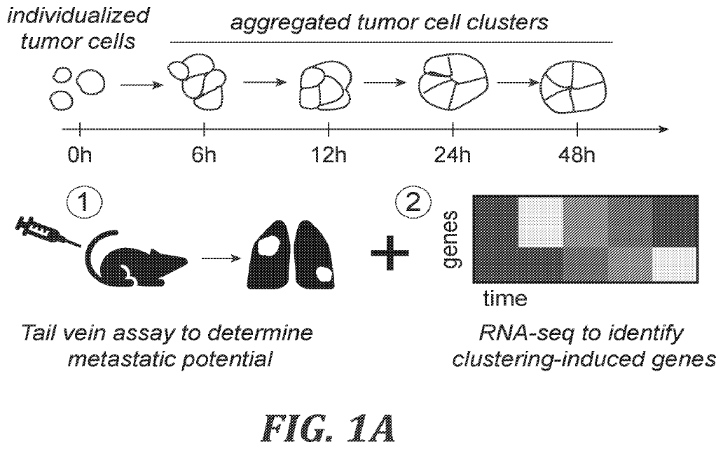

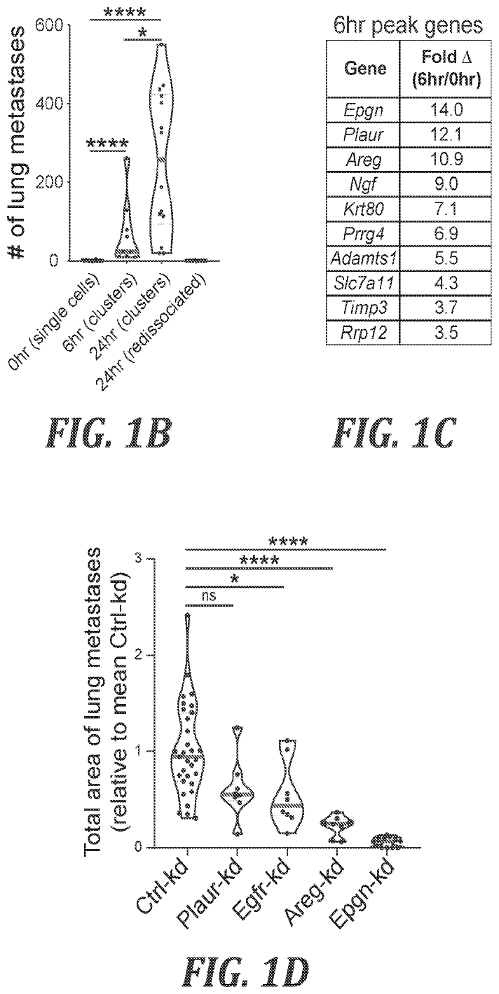

To identify metastasis-promoting signals generated by aggregation, the inventors first developed an experimental system to temporally control cluster formation and monitor downstream molecular events (FIG. 1A). The studies began with an aggressive mouse model of breast cancer, MMTV-PyMT, in which collective invasion and multicellular metastasis have been reported. When MMTV-PyMT tumor organoids were enzymatically dissociated and cultured in suspension, individual tumor cells spontaneously assembled into loosely attached cell aggregates by 6 hrs and more compact clusters by 24 hrs, with a mean number of cells per cluster of 4.4 (SD±4.2) and 5.4 (SD±5.2), respectively. MMTV-PyMT membrane-Tomato labeled tumor organoids were dissociated to single cells, then four input suspensions were generated: single cells (aggregated for 0 hrs), cells aggregated for 6 hrs, cells aggregated for 24 hrs, and cells aggregated for 24 hrs then re-dissociated back to single cells. Equal cell numbers of each suspension were injected into immunocompromised NSG mice. 3 weeks later, loosely attached (6 hr) clusters formed 140-fold and compact (24 hr) clusters formed 536-fold more lung metastases than an equal number of single cells (FIG. 1B). Well-organized (24 hr) clusters re-dissociated back into single cells were negligibly metastatic (FIG. 1B), indicating that metastatic potential in this system specifically requires multicellular organization.

Further, it was observed that both single cells and clusters were trapped in the lungs of mice shortly after tail vein injection. But over consecutive days, clusters persisted while single cells were largely cleared. A small number of single cells persisted in the lungs at 3 wks post-injection but micrometastases were not detected, in contrast to the large number of macrometastases in cluster-injected conditions (FIG. 1B). To assess metastatic potential in organs other than the lung, mTomato-PyMT single cells or clusters were injected into the left cardiac ventricle of NSG mice. Intracardiac injection of clustered tumor cells generated significantly more metastases to multiple organs including the brain, lungs, and liver than injection of single cells.

Concordant with these in vivo observations, ex vivo experiments in 3D culture revealed that MMTV-PyMT tumor cell clusters have >4.7-fold higher outgrowth than single cells, measured as change in area during time lapse imaging. Proliferation was higher in doublets compared with single cells, and highest in larger clusters with tumor cells directly contacting multiple neighbors. To assess differences in apoptosis, mixed populations of single cells and clusters embedded together in 3D basement membrane-rich gels were monitored in the presence of a caspase 3/7 fluorescent biosensor. MMTV-PyMT clusters had 3.5-fold higher survival than single cells after 96 hrs. Importantly, 8 of 10 individual human breast cancer tumor specimens, including those from hormone receptor-positive and triple-negative primary tumors and metastases to brain and bone, had 2.5 to >70-fold higher survival as clusters. Together, these data establish a system to dissect the metastasis-promoting signals downstream of tumor cell cluster formation.

The Growth Factor Epigen is the Most Induced Gene Upon Clustering and is Required for Efficient Metastatic Outgrowth in the Lung.

To identify molecular differences between single cells and clusters that underlie their disparate metastatic potentials, the inventors performed an RNA-seq time-course of MMTV-PyMT cells throughout their aggregation from single cells (0 hrs after dissociation) to highly metastatic clusters (6 to 48 hrs after dissociation) (FIG. 1A). Significant differences in gene expression between single cells and clusters and sequential peaks of gene expression at different stages of aggregation were observed. Importantly, gene ontology analysis revealed sequential upregulation of different biological pathways: at 0 hrs, HIF1 and interferon stress signaling pathways; at 6 hrs, wound healing and MAPK-associated genes; at 12 hrs, ribosome biogenesis; and at 24 and 48 hrs, genes involved in cell cycle and fatty acid metabolism.

Analysis of early clusters (6 hrs) compared to single cells revealed that clustering rapidly induced expression of low-affinity EGFR ligands epigen and amphiregulin (FIG. 1C). Epigen was the #1 most differentially expressed of all mRNAs by gene level analysis (Table S3). Compared to high-affinity ligands like EGF, low-affinity EGFR ligands are associated with prolonged signaling and altered effector responses. uPar (Plaur) which signals in part by ligand-independent EGFR activation was also highly induced in clusters. Therefore, it was hypothesized that growth factor production and EGFR activation support the superior metastatic colonization of clusters.

To test the role of EGFR associated genes in collective metastatic outgrowth, lentiviral RNAi was used to deplete Egfr, Epgn, Areg, and Plaur in MMTV-PyMT tumor cell clusters. Knockdowns were validated by qPCR. Importantly, knockdowns did not disrupt the ability of tumor cells to aggregate into clusters, supporting the hypothesis that these factors act downstream of clustering. Knockdown of Egfr, Epgn, Areg, and Plaur all suppressed outgrowth ex vivo in 3D culture. For each condition, equal numbers of cells were injected by tail vein. 3 weeks later, significant reduction in metastatic burden relative to control by Egfr-kd, Areg-kd, and Epgn-kd was observed (FIG. 1D).

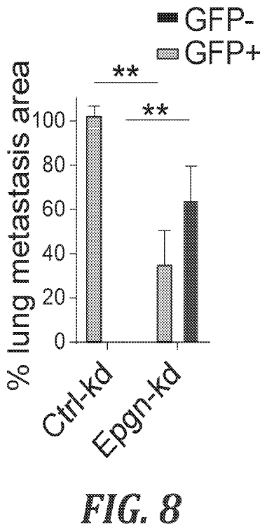

Strikingly, Epgn-kd reduced metastatic outgrowth in the lungs by >94%, and strongly reduced the size but not total number of metastases in the lungs. Further, lung metastases had greatly reduced reporter GFP expression in Epgn-kd but not non-targeting Ctrl-kd cluster injected mice, indicating selective pressure to escape from epigen knockdown (FIG. 8). Taken together, these data indicate that efficient metastatic outgrowth depends on epigen expression.

Epigen Suppression Switches Tumor Cell Clusters from a Proliferative State to a Migratory State.

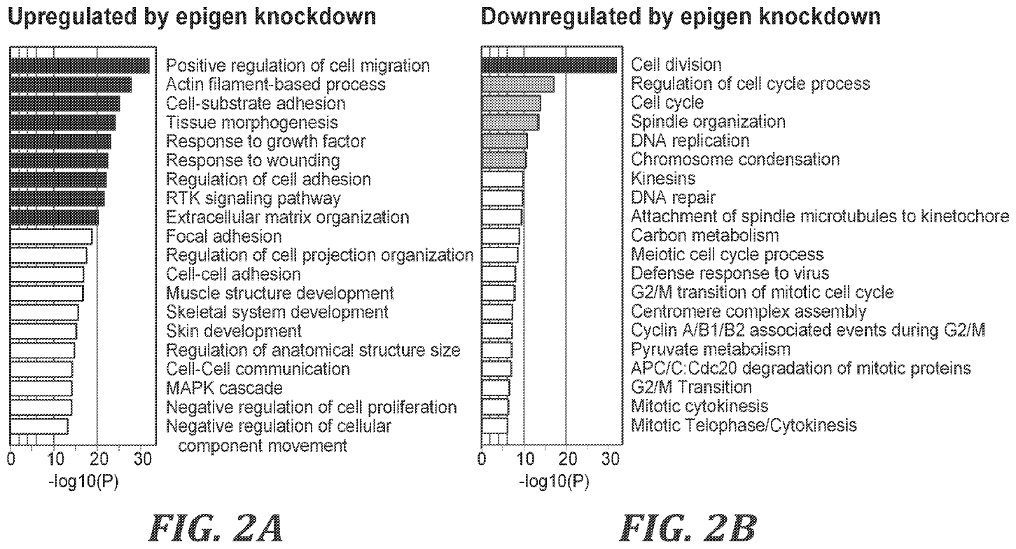

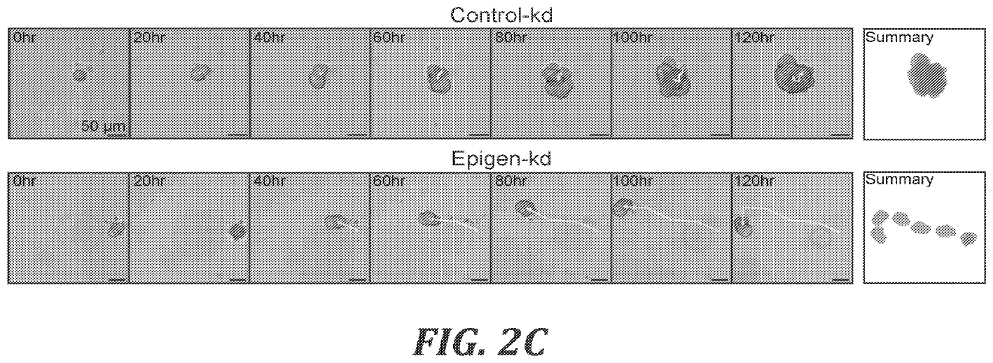

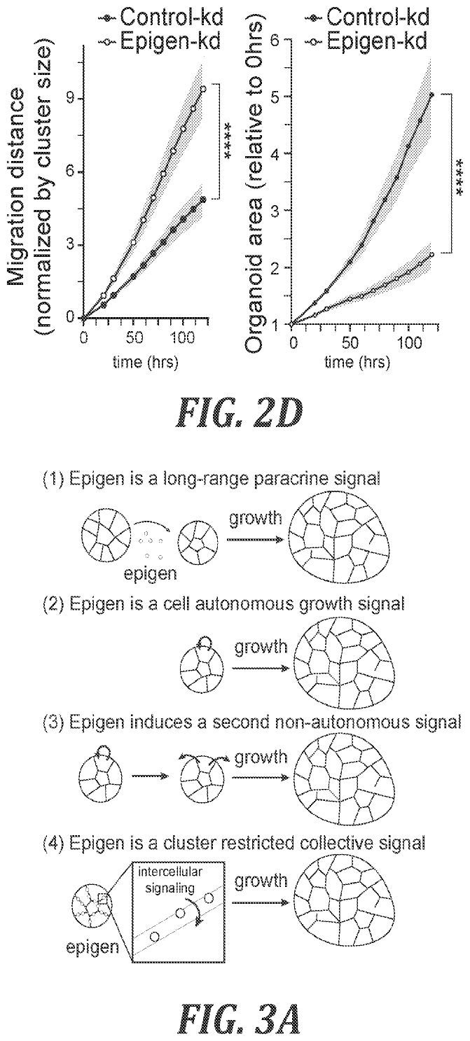

The inventors sought to determine the genes and biological processes regulated by epigen expression. RNA sequencing of Ctrl-kd and Epgn-kd MMTV-PyMT clusters aggregated for 24 hrs revealed that Epgn-kd clusters markedly downregulate cell cycle genes and upregulate genes involved in cell migration (FIG. 2A). Comparing these results with the sequential gene expression patterns observed during cell aggregation, it was found that genes upregulated by Epgn-kd were most often genes that peaked early during aggregation at 6 or 12 hrs. Genes downregulated by epigen knockdown were most often genes that peaked later at 24 or 48 hrs of aggregation. These data indicated that Epgn-kd clusters were in a transcriptional state more closely resembling nascent, non-proliferative clusters.

Analysis of invasion and adhesion related gene-sets revealed that knockdown of epigen induces expression of genes in the cadherin, desmosome. tight junction, and matrix metalloproteinase families Epigen knockdown also induced expression of EMT-associated genes including Fn1, Zeb1, Tnc, and Snai2. Nonetheless, Epgn-kd organoids showed persistent expression of epithelial genes including Epcam and Cdh1, modest induction of cytokeratins Krt5 and Krt14, which are associated with basal/myoepithelial cells and invasive leader cells, and marked induction of Krt17, which is associated with epithelial wound healing and migration.

To functionally test the effects of epigen suppression on collective outgrowth and migration, Ctrl-kd and Epgn-kd clusters were embedded in 3D basement membrane-rich gels and monitored by time-lapse microscopy (FIG. 2B). Compared with Ctrl-kd clusters, Epgn-kd clusters showed markedly increased migration through the 3D matrix and significantly reduced outgrowth (FIGS. 2C-D) and proliferation. Consistent with retained mRNA expression of Cdh1, Krt14, and Krt17, Epgn-kd clusters migrated not as individual cells but as E-cad+/K14+/K17+ clusters. It was further confirmed that a second epigen shRNA also significantly reduced epigen expression and outgrowth, and that outgrowth was significantly rescued by transduction with an shRNA-resistant human epigen construct. Taken together, these data reveal that epigen expression supports the transition of clustered tumor cells from a collective migratory to proliferative state.

Epigen Acts as a Collective Signaling Factor Shared Non-Cell-Autonomously within Clusters

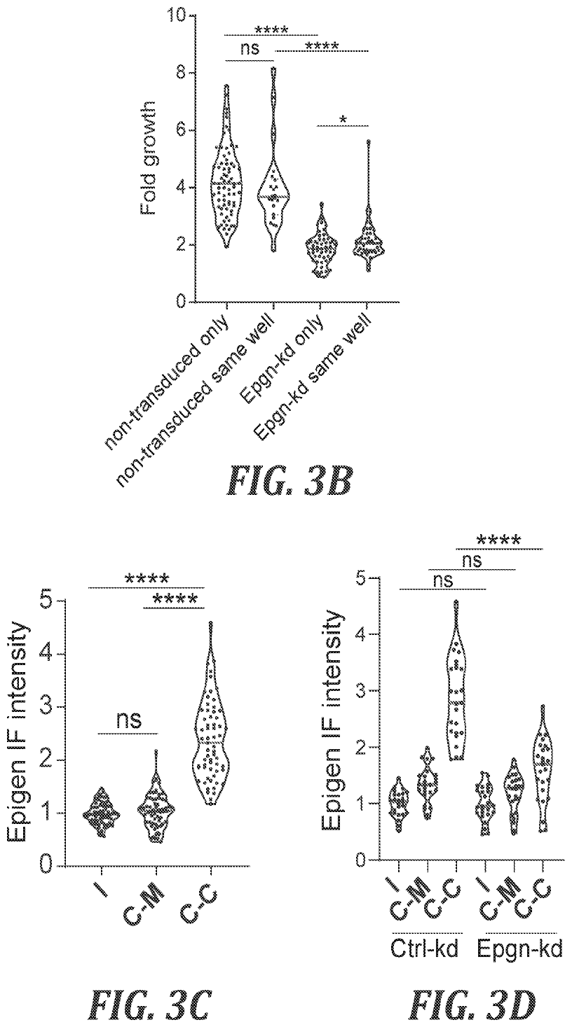

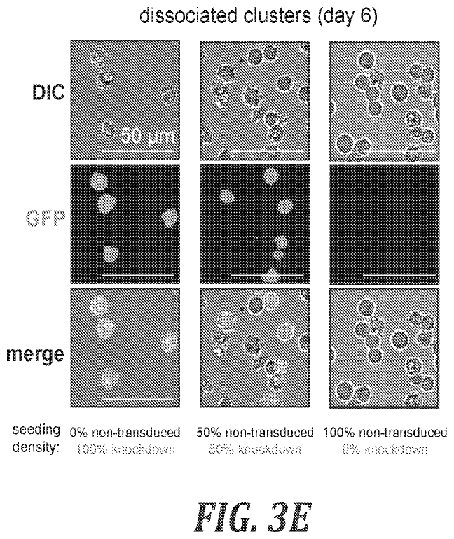

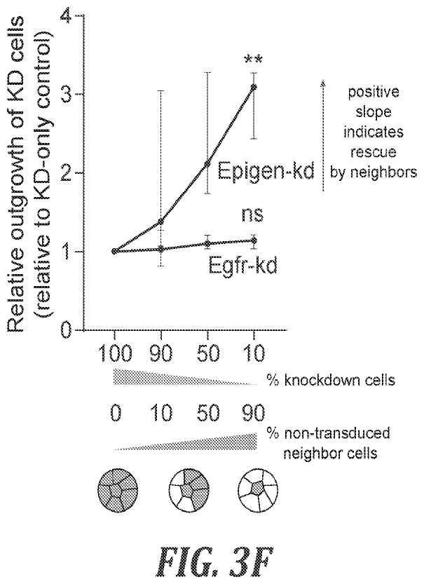

Without wishing to be bound by theory, there are several possible mechanisms by which epigen could induce cluster outgrowth (FIG. 3A). Epigen could be secreted from clusters into their local microenvironment, facilitating long-range paracrine signaling (model 1). Epigen could act in an autocrine manner, inducing proliferation in the same cell that expresses it (model 2). Epigen could induce paracrine growth factors downstream of receptor activation (model 3). Or, epigen could be a non-cell-autonomous growth factor whose signaling range is restricted to each tumor cell cluster (model 4).

If epigen acts as a long-range secreted molecule with no local restriction (model 1), then co-culture with non-transduced clusters should partly rescue the outgrowth defect of nearby Epgn-kd clusters. However, time-lapse imaging indicated that Epgn-kd clusters co-cultured with non-transduced clusters had similar outgrowth rates as those cultured in 3D gels alone (FIG. 3B). These data place limits on the signaling distance of epigen and indicate that epigen acts primarily as a short-range outgrowth signal.

Consistent with a model of short-range signaling, immunofluorescence revealed that epigen is largely restricted to intercellular areas in clusters from the MMTV-PyMT (FIG. 3C), MMTV-Neu, and C3(1)-TAg mouse models of breast cancer. This intercellular enrichment was depleted upon epigen knockdown (FIG. 3D). Epigen protein contains a transmembrane domain but is cleaved by ADAM17 to release the soluble portion from the plasma membrane. mTomato-PyMT tumor cell clusters transduced with a mGFP tagged epigen construct likewise had enriched GFP signal at intercellular regions. These findings are consistent with a short-range, intercellular growth factor.

Having excluded long-range secretion of epigen, the inventors next sought to test different cluster-restricted models of epigen signaling (models 2 through 4). If epigen ligand is shared non-cell-autonomously by neighboring cells, it was hypothesized that adding epigen-expressing cells to a cluster should rescue the outgrowth of adjacent epigen deficient cells. Epgn-kd cells were mixed with non-transduced cells to form mosaic clusters with varying proportions of cells from each population (FIGS. 3E-3F). After 6 days, clusters were dissociated and the number of Epgn-kd cells was counted to assess their outgrowth in each condition relative to the starting cell number. Mixing non-transduced cells and Epgn-kd clusters at a 9:1 ratio increased the outgrowth of Epgn-kd cells ˜3-fold higher than when cultured alone (FIG. 3G). Further supporting this finding, Epgn-kd cells aggregated with Ctrl-kd cells at a 1:1 ratio migrated markedly less than pure Epgn-kd clusters.

These data showed that epigen can induce the outgrowth of neighboring cells non-cell-autonomously. In support of this hypothesis, treatment with recombinant epigen induced outgrowth in a dose-dependent manner. However, an alternative explanation is that epigen does not directly promote outgrowth but rather induces expression of another growth signal that is transmitted laterally to neighboring cells. To test this, mosaic mixing experiments were performed with mixtures of non-transduced cells and cells transduced with a shRNA targeting Egfr, the known receptor for epigen. Egfr-kd cells in mosaic clusters did not grow significantly more than when cultured as pure Egfr-kd clusters (FIG. 3G), demonstrating that non-transduced cells do not produce a second signal that can rescue Egfr-kd outgrowth. Together, epigen's intercellular enrichment, short-range restriction, and non-cell-autonomous activity all indicate that epigen acts within clusters as a shared, collective growth signal (model 4).

Secreted Epigen is Stored and Concentrated within Largely Impermeable Intercellular Microlumina

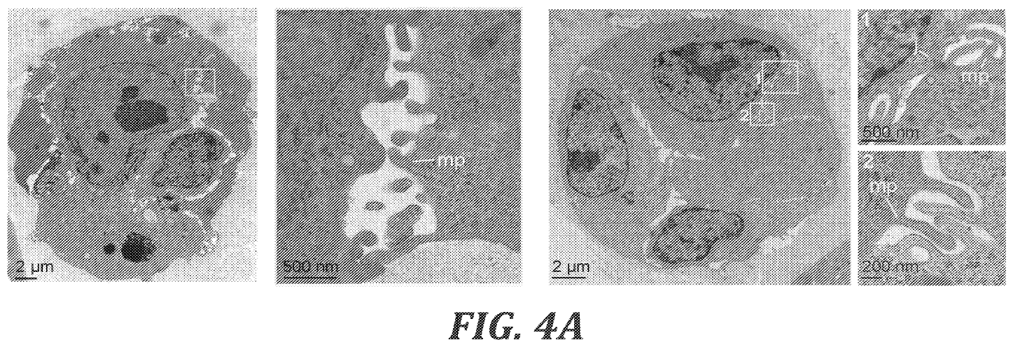

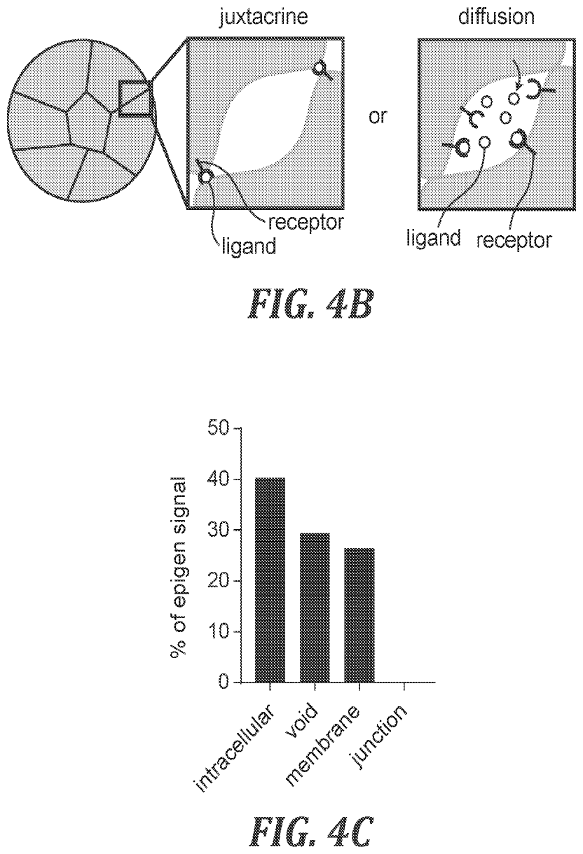

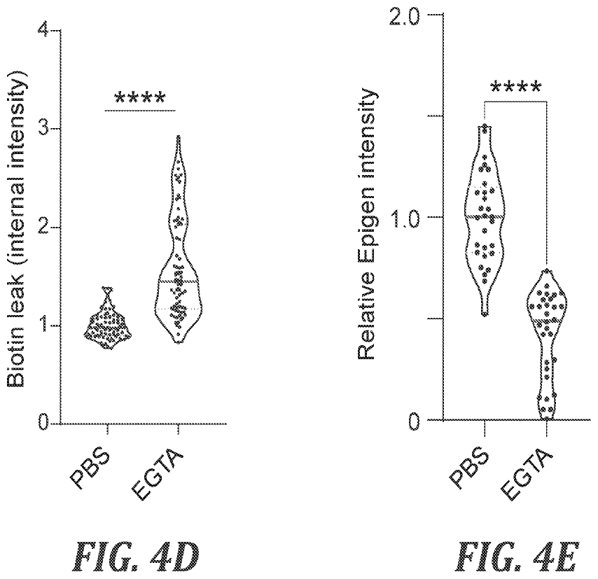

To characterize the physical properties of the epigen collective signaling compartment, MMTV-PyMT clusters were examined by transmission electron microscopy (TEM). Surprisingly, it was observed that intercellular membranes, rather than being closely apposed, were often separated by hollow cavities. These cavities were typically punctuated and sealed with electron-dense cell-cell junctions, and frequently lined by interdigitating microvilli-like protrusions (FIG. 4A). These hollow, intercellular cavities are referred to herein as “microlumina.”

Without wishing to be bound by theory, it was hypothesized that the microlumina observed in tumor cell clusters could function as hubs to concentrate soluble signaling molecules like epigen in intercellular cavities (FIG. 4B). Alternatively, epigen could localize to areas of direct cell-cell contact and activate EGFR in a membrane-restricted juxtracrine manner. Consistent with the first model, stimulated emission depletion microscopy revealed robust concentration of epigen within intercellular spaces (FIG. 4C). The intercellular localization of epigen was not restricted to areas expressing the apical marker Muc1, suggesting that these are not apically polarized lumens. Immunoelectron microscopy additionally confirmed the presence of epigen ligand within microlumenal cavities and along microlumenal membranes but not at direct cell-cell junctions. Together with the non-cell-autonomous effects of epigen on cluster outgrowth, these findings suggest that epigen diffuses within shared microlumina between clustered cells.

The accumulation of epigen within microlumina implied a mechanism for preventing ligand diffusion and signal reduction. To characterize the permeability of tumor cell clusters, a technique used to differentially label apical and basal proteins was adapted. Clusters were treated with a cell impermeable biotin (sulfo-NHS-biotin, 0.4 KDa) which labels membrane surface proteins but is excluded from the cytosol. At baseline, biotin was excluded from cell-cell junction membranes within MMTV-PyMT clusters and predominantly labeled the cell-matrix junction membranes (FIG. 4D). In the presence of calcium chelation, which disrupts cell-cell junctions, biotin was able to leak into intercellular spaces (FIG. 4D). Disruption of junctions by Latrunculin A or ochratoxin A also allowed biotin to leak between clustered cells.

Immunofluorescence of MMTV-PyMT clusters after calcium chelation also revealed a marked loss of epigen signal in clusters (FIG. 4E). Importantly, it was estimated that the microlumenal concentration of epigen is >5000-fold higher than the measured extra-cluster concentration (223 vs. 0.04 ng/mL). Epigen knockdown did not alter microlumina morphology, area, or perimeter, indicating that epigen expression is not required for microlumina formation. Thus, junctional sealing of microlumina facilitates a high local concentration of epigen between clustered cells.

Having localized epigen to the microlumenal compartment, next the dominant receptor tyrosine kinase pathways activated in tumor cell clusters were determined. To assess receptor tyrosine kinase activation in an unbiased manner the inventors performed protein array analysis of 39 different receptor tyrosine kinases in single cell and 6 hr-cluster PyMT lysates. ERBB family members pTyr-EGFR and pTyr-HER2, which can heterodimerize with EGFR, were the most cluster-upregulated phospho-RTKs. Additionally, robust induction of EGFR effectors pERK1/2 and pAKT was observed by western blot in clustered tumor cells compared with individual tumor cells. These examples show that cluster formation is associated with phosphorylation and activation of EGFR and HER2, and that this synergistic induction is not apparent in cells that remain individualized. In addition, the inventors noted an enrichment of pEGFR and HER2 localization at intercellular areas between clustered tumor cells. Using immunoelectron microscopy, it was confirmed that pEGFR and Her2 localized primarily along microlumenal membranes as opposed to within microlumenal cavities, as would be expected of transmembrane receptors. These data show that microlumina form a productive compartment for epigen-EGFR signaling.

Epigen Suppression Reduces Both Primary Tumor Outgrowth and Spontaneous Metastasis to the Lung

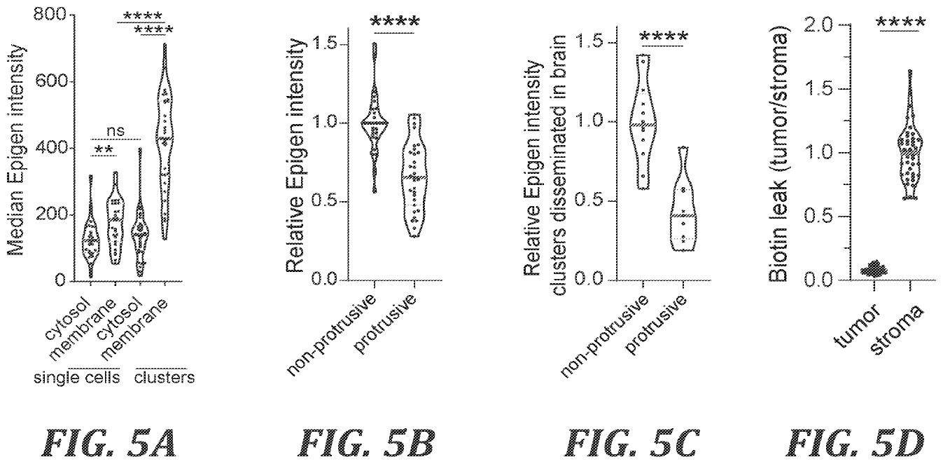

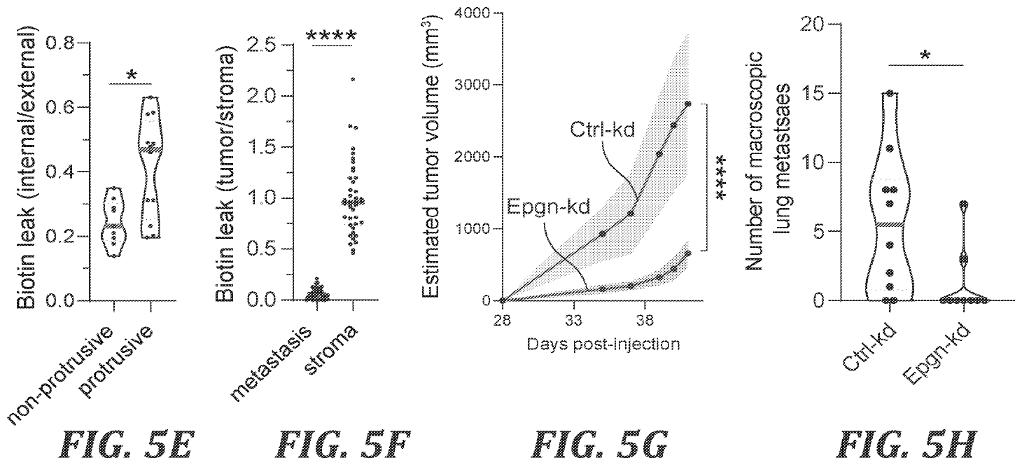

Having identified the site and mechanism of epigen signaling ex vivo, the inventors next assessed epigen expression and microlumina formation during metastasis. To assess epigen expression in primary tumors, MMTV-PyMT mTomato+ clusters were transplanted into the mammary fat pads of NSG mice to generate primary tumors that could be distinguished from non-fluorescent host tissue. Intercellular epigen was highly expressed in primary tumors and frequently in locally disseminated tumor cell clusters (FIG. 5A-B). Likewise, distant metastases to the brain, liver, and lung consistently expressed intercellular epigen (FIG. 5C), pEGFR, and HER2.

In contrast, epigen expression was limited in locally disseminated single cells (FIG. 5A) and in the surrounding tissue microenvironment of breast cancer metastases (FIG. 5C) which was confirmed in normal human tissues by RNA-seq. Importantly, the inventors observed that locally disseminated tumor cell clusters were categorized by two distinct morphologies: protrusive and rounded/non-protrusive. Strikingly, highly protrusive clusters had markedly lower epigen expression than their non-protrusive, rounded counterparts (FIG. 5B). Pan-cytokeratin, keratin-14, and keratin-17 staining confirmed that these highly protrusive tumor strands were of epithelial origin, not contaminating stromal cells. Likewise, highly protrusive clusters locally disseminated from brain metastases arising from intracardiac injection of mTomato-PyMT clusters expressed epigen at lower levels than their non-protrusive counterparts (FIG. 5C). These findings reveal that epigen expression is regulated during metastatic dissemination, with lowest expression in protrusive locally disseminated clusters, and highest expression in phases associated with tumor outgrowth.

To characterize microlumina in vivo, primary MMTV-PyMT tumors were then subjected to TEM. Electron microscopy of these samples revealed extensive intercellular microlumina with similar microvilli-like protrusions and cell-cell junctions as observed ex vivo. When MMTV-PyMT tumors were exposed to sulfo-NHS-biotin for 30 minutes prior to fixation, biotin was found extensively in the tumor-adjacent stroma but was largely excluded from epithelial tumor cell nests (FIG. 5D). Exposure of primary tumors to biotin further revealed an increase in biotin permeability in highly protrusive strands compared to non-protrusive tumor cell clusters (FIG. 5E). To assess microlumenal permeability at secondary sites, tumor cell clusters were injected by tail vein into NSG mice. Two weeks later, lungs were harvested and exposed to NHS-sulfo-biotin. Importantly, lung metastases were far more biotin impermeant than the adjacent lung tissue (FIG. 5F). These results indicate that microlumina are junctionally tight in primary tumors and distant metastases, which the inventors observed have high epigen expression, but junctionally leaky in protrusive locally disseminated clusters, which have low epigen expression.

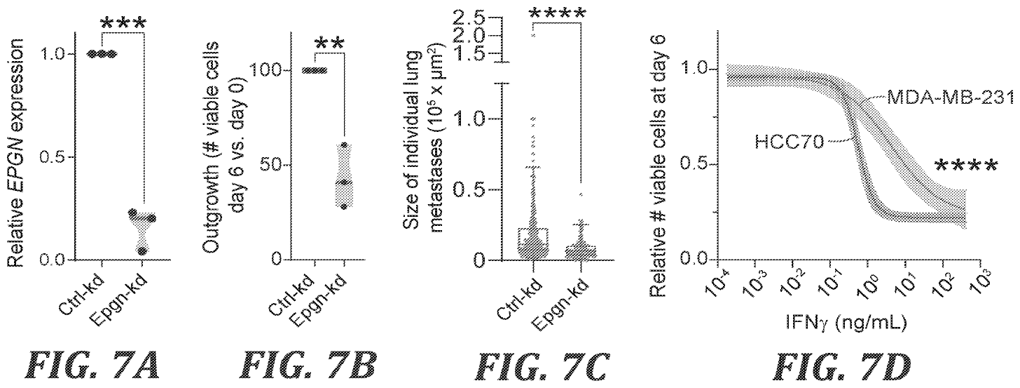

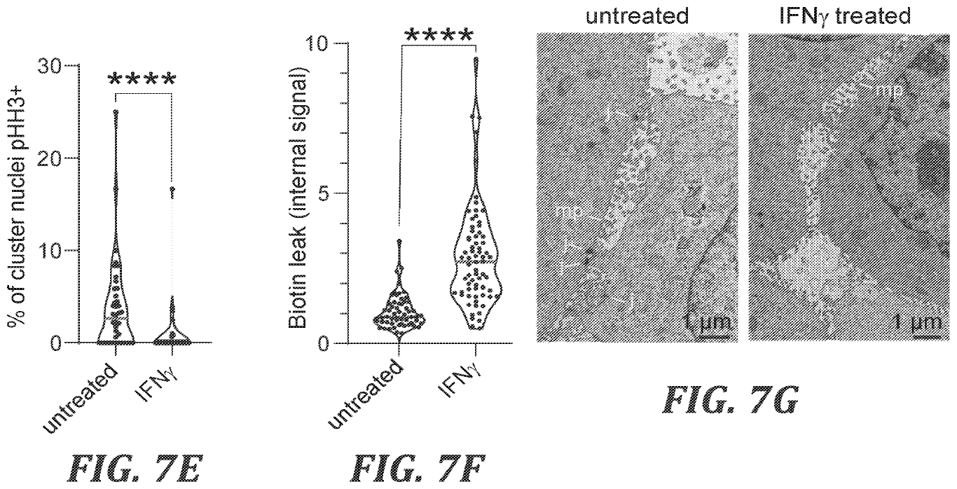

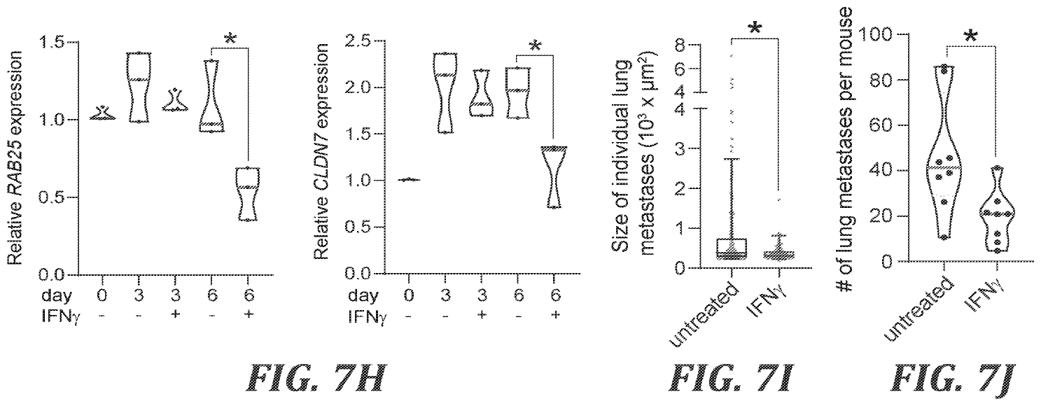

To assess the functional role of epigen during metastatic progression, equal cell numbers of Ctrl-kd and Epgn-kd clusters were orthotopically transplanted into the mammary fat pads of immunocompromised mice. Over the ensuing 6 weeks, the inventors observed significantly slower outgrowth of Epgn-kd primary tumors compared to Ctrl-kd tumors (FIG. 5G). Analysis of the tumor-stroma border of these tumors showed a modest increase in locally disseminated clusters in Epgn-kd tumors compared to Ctrl-kd. Importantly, Epgn-kd tumors also formed fewer spontaneous macrometastases to the lung than Ctrl-kd tumors (FIG. 5H). Epgn-kd metastases were significantly less proliferative and smaller than Ctrl-kd lung metastases. Though the number of visible macrometastases was significantly different (FIG. 7L), when micrometastases were quantified there was no significant difference between Ctrl-kd and Epgn-kd mouse lungs similar to what was observed after tail vein injection of Epgn-kd clusters. Epgn-kd orthotopic tumors also lost expression of the shRNA-GFP vector at much higher frequencies than Ctrl-kd tumors, indicating selective pressure on tumor cells to revert to Epgn-high phenotypes in order to permit outgrowth as noted after Epgn-kd tail vein (FIG. 8). Thus, epigen suppression reduces primary tumor and metastatic outgrowth, while increasing local dissemination of clusters, consistent with the findings in ex-vivo 3D culture.

High Epigen Expression and Microlumina Formation are Associated with the Basal-Like 2 Subgroup of Triple-Negative Breast Cancer

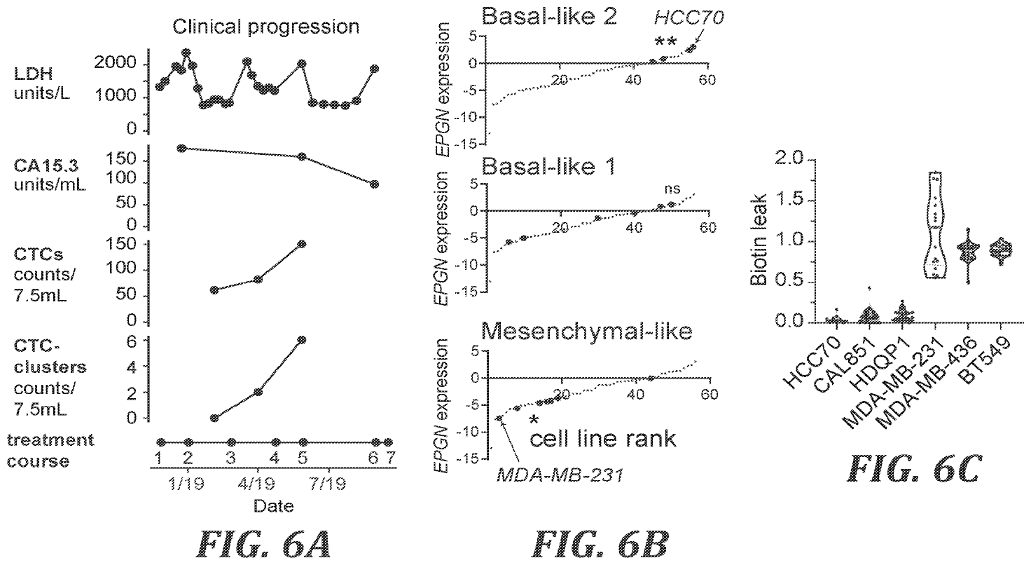

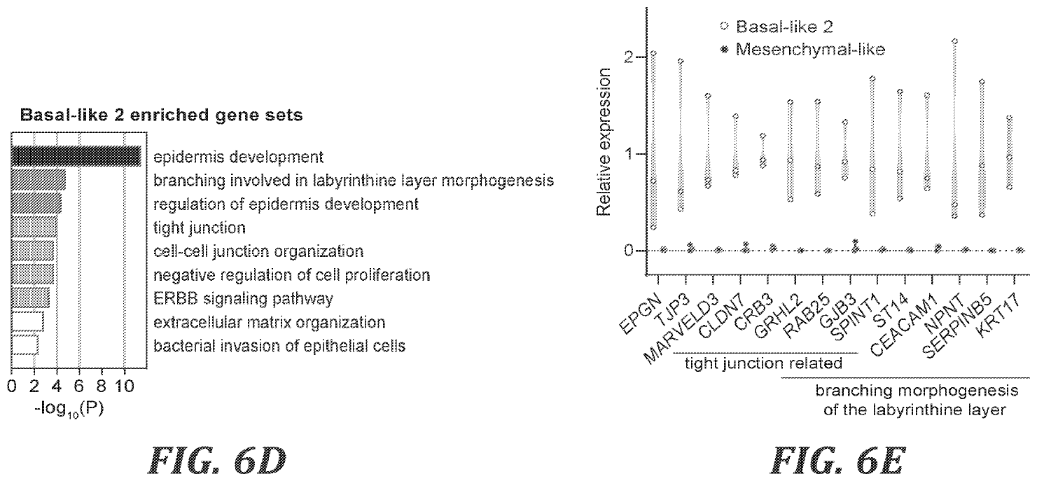

The generality of the above findings to human breast cancer was investigated. As a first approach, tumor cell clusters were collected from a metastatic breast cancer patient recruited to an IRB approved study at the Fred Hutchinson Cancer Research Center (Seattle, Wash.). At the time of enrollment, the patient was a 52-year-old female with ER+/PR+/HER2-breast cancer that was metastatic to orbit, lung, liver, and bone. Using the Rarecyte platform, CTC clusters were detected at the second and third time points, coinciding with clinical evidence of disease progression (FIGS. 6A-B). Palliative paracentesis revealed malignant ascites containing cytokeratin-positive/CD45-negative tumor cell clusters. The large volume of ascites containing many tumor cell clusters enabled more detailed molecular analyses Immunofluorescence of collected clusters demonstrated phosphorylated EGFR at intercellular contacts and enriched epigen expression relative to adjacent CD45+ cells. Further, TEM revealed microlumina with microvilli-like protrusions in ascites-derived tumor cell clusters. Malignant ascites were also collected from a second patient; a 63-year-old female with metastatic ER+/PR+/HER2-breast cancer. At the time of paracentesis, she was treated with letrozole and abemaciclib, and had metastatic disease to bone, liver, chest wall, stomach, and peritoneum. Similarly, tumor cell clusters collected from this patient demonstrated intercellular pEGFR and epigen expression as well as intercellular microlumina. These clinical observations support the disease relevance of microlumenal signaling for in vivo metastasis.

As a second approach, epigen in two RNA-seq surveys of human breast cancer cell lines was examined. The cell lines were sorted by EPGN mRNA expression and ER/PR/HER2 receptor status. Triple-negative cell lines were further separated by previously described subgroups basal-like 1 (BL1), basal-like 2 (BL2), mesenchymal-like (M-like), and luminal androgen receptor (LAR). A strong association between high EPGN expression and the basal-like 2 subgroup was observed, whereas mesenchymal-like breast cancer cell lines had the lowest EPGN expression. Triple-negative breast cancers frequently overexpress EGFR which is associated with poorer prognosis. BL2 triple-negative breast cancers in particular have increased growth factor signaling and basal/myoepithelial gene expression and have been reported to have between 0 to 22% response rates to chemotherapy. In addition, the highest epigen expressing HER2+ cell lines were associated with the recently described basal-like (HER2E) subset of HER2+ cell lines. Likewise, basal-like HER2+ patient tumors have increased expression of RTKs including EGFR and HER2 relative to luminal HER2+ tumors. These data demonstrate that high epigen expression is linked to high-risk subtypes of human breast cancer.

In addition, TEM revealed considerable morphological differences between three BL2, epigen-high cell lines (HCC70, CAL851, and HDQP1) and three M-like, epigen-low cell lines (MDA-MB-231, MDA-MB-436, BT549). BL2 cell clusters contained microlumina with extensive microvilli-like protrusions, frequently sealed by electron-dense junctional complexes. In contrast, M-like cell clusters were more loosely organized and largely lacked junctionally-sealed intercellular spaces. Consistent with these two distinct morphologies, BL2 cell clusters were highly biotin-impermeable, whereas biotin readily leaked into the intercellular spaces of M-like clusters (FIG. 6C). Importantly, transcriptional analysis of these 6 cell lines revealed that BL2, epigen-high lines were enriched for genes related to epithelial development and migration; branching morphogenesis of the placental labyrinthine layer; and tight junction genes including CLDN7, MARVELD3, and the transcription factor GRHL2 (FIG. 6D-E). Mesenchymal-like cell lines, on the other hand, were enriched for genes related to collagen metabolism and FGFR signaling. These data show that epigen enrichment and microlumina occur in aggressive metastatic breast cancers, and reveal large differences in microlumina formation, junctional permeability, and morphogenesis-related gene expression between BL2 and M-like cells.

HCC70 Outgrowth Depends on Epigen Expression and is Exquisitely Sensitive to IFNγ which Induces Microlumen Permeability

The highest EPGN expressing cell line in both RNA-seq datasets was the BL2 line HCC70. Notably, when compared to a transcriptomic dataset of metastatic solid tumors (MET500), HCC70 cells modeled the gene expression of metastatic basal-like breast cancers more closely than other tested cell lines. HCC70 clusters highly expressed epigen, pEGFR, and the tight junction protein occludin and as noted above, were largely biotin-impermeable with sealed microlumina lined by extensive microvilli-like protrusions (FIG. 6C).

Importantly, EPGN knockdown in HCC70 cells significantly reduced their outgrowth ex vivo (FIGS. 7A-B). When grown in 2D cell culture as monolayers, no significant differences in 2D migration or outgrowth were observed. Transduction with an EPGN sgRNA as an independent method of epigen suppression also significantly reduced HCC70 cluster outgrowth relative to control. To assess epigen's role in metastatic outgrowth, Epgn-kd HCC70 cell clusters were injected by tail vein into immunocompromised NSG mice. 3 weeks later, the Epgn-kd clusters had formed significantly smaller metastases than Ctrl-kd clusters (FIG. 7C). Thus. epigen supports metastatic outgrowth in human basal-like 2 HCC70 cells. This shows that epigen dependence and microlumina formation occur in both mouse and human models of breast cancer.

The existence of microlumina in human models suggested that this structure could be targeted to reduce collective signaling during metastasis. Previous studies have demonstrated that IFNγ, a cytokine FDA-approved for use in patients, can disrupt epithelial permeability and enhance paracellular diffusion independent of apoptosis induction. During infection, IFNγ induces increased epithelial flux to amplify inflammatory signaling and immune response. In the RNA-seq data used herein, interferon-associated genes were induced in single cells, and repressed upon aggregation, suggesting a role for this pathway in multicellular compared with individual transcriptional states. Thus, IFNγ could weaken the barriers protecting microlumina and inhibit cluster outgrowth.

Consistent with the above, HCC70 tumor cell clusters were highly sensitive to IFNγ treatment (IC50=0.58 ng/mL, IC75=0.97 ng/mL) and showed markedly reduced outgrowth (FIG. 7D). A strong suppression of proliferation by IFNγ but not increased cell death in HCC70 clusters was observed (FIG. 7E). Importantly, IFNγ treatment increased intercellular permeability in HCC70 clusters by biotin-leak assay (FIG. 7F) and showed increased microlumenal spacing and decreased electron-dense junctions by TEM (FIG. 7G). On a molecular level, qPCR of HCC70 clusters treated with IFNγ revealed a significant reduction in RAB25, a tight junction regulator, and CLDN7, a component of tight junctions and modulator of paracellular permeability. Conversely, the epigen-low mesenchymal TNBC cell line MDA-MB-231 was considerably less sensitive to IFNγ treatment than HCC70 (IC50=4.24 ng/mL, IC75=22.3 ng/mL) (FIG. 7D), expressed RAB25 and CLDN7 at markedly lower levels than HCC70 cells, and was highly biotin-permeable in both the absence or presence of IFNγ treatment.

In addition, the effects of IFNγ treatment on metastatic colonization in vivo were tested. HCC70 clusters were pre-treated with or without low-dose IFNγ (2 ng/mL). After 6 days of treatment, equal viable cell numbers of clustered cells were injected by tail vein into NSG mice. At 3 weeks, HCC70 metastases were identified by staining lung sections with a human specific CD298 antibody. IFNγ pre-treated HCC70 clusters formed significantly smaller and fewer lung metastases than untreated controls (FIGS. 7I-J). Taken together, these data showed that microlumen-opening agents like IFNγ can impair the collective metastatic outgrowth of triple-negative breast cancer cells.

Discussion:

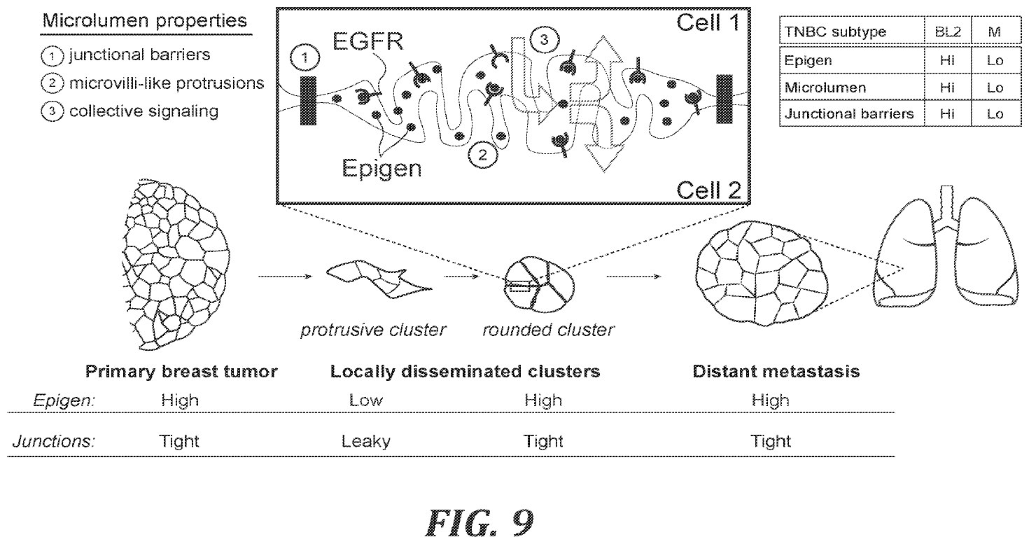

The identification of factors promoting metastasis is a pivotal problem. Importantly, the formation of tumor cell clusters is associated with markedly increased metastatic efficiency. Although cell adhesion is a necessary step for cluster formation, the downstream signaling events that drive the outsized metastatic potential of clusters relative to individual cells have remained unclear. In the present disclosure, the inventors have identified a collective signaling mechanism arising from the multicellular organization of tumor cell clusters (FIG. 9). Here the inventors found that multicellular clusters form microlumina—intercellular compartments sealed from the external environment—that drive breast tumor outgrowth at both primary and metastatic sites. It was demonstrated that microlumina accumulate a diffusible growth factor, epigen, at a local concentration much higher than the outside environment, and that epigen acts as a non-cell-autonomous collective signaling factor.

It was observed that epigen and microlumen permeability are regulated during metastasis; with peak epigen levels and junctional tightness associated with phases of outgrowth, and reduced epigen levels and junctional leakiness in highly protrusive locally disseminated clusters. The ex vivo and in vivo functional studies disclosed herein also support dependence on an epigen signaling axis that regulates switching between proliferative and migratory modes during collective metastasis. The ability of metastatic cancer cells to transit through the stages of metastasis is understood to involve multiple regulatory factors, both internal to the cancer cell as well as arising from interactions with its tumor microenvironment. The studies disclosed herein reveal an additional dimension: the shared intercellular exchange of growth factors between clustered tumor cells. In this way, tumor cell clusters construct their own internal microenvironment and produce their own collective signals to drive metastatic outgrowth.

Collective signaling is supported by the distinctive physical architecture of microlumina. The cell-cell junctions that seal either end of microlumina gate the entrance and exit of certain molecules, securing the molecular composition of this signaling compartment. The microvilli-like protrusions that line these spaces generate a tortuous environment, with high surface area available for intercellular interactions. Because high tortuosity causes the diffusions paths of soluble species to deviate from straight lines this can reduce diffusivity and increase signaling. Thus, the increased surface area and tortuosity generated by microlumenal microvilli-like protrusions, combined with the restricted diffusion of epigen through junctional contacts, creates a 3D environment conducive to focused signaling. Recent findings in other experimental systems have described the impact of 3D topology on critical signals during development and in generating synthetic biological systems. This disclosure supports the parallel importance of topology in shaping signaling during metastasis, in this case arising from the multicellular organization of tumor cell clusters.

Importantly, the findings disclosed herein support the human disease relevance of microlumina in metastatic tumor cell clusters. The inventors directly observe microlumenal structures in freshly isolated tumor cell clusters from metastatic breast cancer patients with malignant ascites. Further, the studies disclosed herein demonstrate that aggressive triple negative breast cancer cells, specifically the basal-like-2 subtype, are associated with high epigen expression and dependence on microlumenal signaling. A wide variety of tumor types are collectively organized and show a propensity to disseminate as clusters. It therefore seems likely that systematically interrogating the presence and composition of microlumina in other cancer types could uncover additional collective signaling mechanisms. Likewise, microlumenal interrogation could reveal dynamic expression of signaling receptors and microlumenal factors beyond epigen, regulating different steps of collective invasion and multicellular metastasis.

In addition, microlumenal signaling could play an important role in normal development and tissue homeostasis. Ultrastructural studies of normal mammary epithelia have revealed interdigitating structures similar to the microlumina characterized in this study, but whose functional significance has remained unknown. The collective signaling mechanism identified herein, and its regulation during phases of outgrowth and migration, suggest parallels with the proliferation and collective migration of developing mammary terminal end buds. Likewise, the enrichment of genes related to labyrinthine placental morphogenesis in BL2 cancer cells may hint at a primordial developmental role for microlumina that is being co-opted during metastasis, analogous to the co-option of EMT programs in mesenchymal cancer cells. A detailed understanding of the genes regulating microlumenal formation and signaling, in development and disease, could reveal therapeutic targets specifically important for microlumenal signaling. For example, it was found that the cytokine IFNγ strongly disrupts microlumen impermeability and metastatic colonization. Therefore, targeted disruption of microlumenal junctions can suppress outgrowth of collectively metastasizing cancers.

Experimental Model and Subject Details

Animal protocols were approved by the Fred Hutchinson Institutional Animal Care and Use Committee. FVB/N-Tg(MMTV-PyVT)634Mul/J (MMTV-PyMT) were maintained and tumor growth was monitored every 2 days. MMTV-PyMT mice were crossed with ROSA mTomato/mGFP mice to generate mTmG-PyMT mice with mTomato+ cell membranes allowing easy identification when injected into non-fluorescent host mice. For injection experiments immunocompromised NSG mice (NOD.Cg-Prkdc^{scid} Il2rg^{tm1Wjl}/SzJ) were used. Adult female mice were used for all experiments. Deidentified human breast cancer primary or metastatic samples were received from the Cooperative Human Tissues Network and the Seattle Cancer Care Alliance. Tumor samples were processed as previously described to generate organoids. Blood and ascites fluid were collected under a Fred Hutch IRB approved study (FH8649) for longitudinal monitoring of circulating tumor cells in metastatic breast cancer patients.

Method Details

Tail Vein and Intracardiac Injection of NSG Mice

For single cell vs. cluster experiments, mTomato-PyMT organoids were dissociated to single cells at day 0 using Accumax (20 minutes at 37° C.). 200,000 cells in 200 uL DPBS were injected per mouse in the tail vein. To generate clusters, single cells were plated in non-adherent dishes at 150,000 cells/mL in media+2% basement membrane-rich gel (v/v) and then injected as above 6 hr or 24 hrs later into Nod scid gamma (NSG) immunocompromised mice. shRNA knockdown injections were all carried out with 24 hr aggregated clusters. DIC images were taken before injection and assessed to ensure similar number and size of clusters injected between conditions. 3 weeks after tail vein injection mice were euthanized and lungs imaged under a dissecting microscope for quantification of fluorescent (metastatic) area. For intracardiac injections, 100,000 mTomato-PyMT clustered cells were injected into the left ventricle in 100 μL of PBS using a 26 g needle with ultrasound guidance with 2.5% isoflurane anesthesia. 6 weeks later the brain, liver, lung, femur, kidneys, and ovaries were collected. Collected organs were fixed in 4% PFA for 4 hrs, then transferred to 25% sucrose in DPBS overnight at 4° C. before embedding in OCT and storing at −80° C.

Mouse Mammary Tumor Organoid Culture

Organoids were isolated from MMTV-PyMT, C3(1)TAg, or MMTV-Neu mouse mammary tumors as previously described. Briefly, tumors were dissected, mechanically disrupted with a scalpel, and then digested in a collagenase-trypsin solution for 30-60 minutes. Mice were harvested as the largest tumor neared 1.5 cm in diameter. For 3D culture, 100-200 clusters were embedded in 100 uL of growth-factor reduced Matrigel (Corning), the matrigel was allowed to polymerize for 30-60 minutes, then 1 mL of organoid media (DMEM-F12, FGF2, insulin-transferrin selenium, & penicillin/streptomycin) was added. For suspension culture, clusters were cultured in non-adherent dishes in organoid media+2% (v/v) Matrigel (low basement membrane suspension culture). Tips and tubes used to handle organoids were first coated in 2.5% BSA in DPBS to prevent loss of material. To create single cell suspensions, organoids were centrifuged, resuspended in Accumax (Innovative Cell Technologies) for 20 minutes at 37° C., and counted on a hemocytometer to ensure a low number of residual clusters.

Human Cell Line and Patient Tumor Organoid Culture

Human cell lines were cultured in complete media (DMEM or RPMI with penicillin/streptomycin+10% FBS) on tissue-culture treated plates, or in non-adherent plates supplemented with 2% Matrigel (v/v). To assess migration in 2D, 100,000 viable cells in 10 uL media were seeded as a droplet at the center of a Collagen I coated 24 well plate. After 30 minutes at 37° C., 1 mL of complete media was added. Tiled DIC and GFP images were taken 1 day and 6 days after plating. Deidentified human breast cancer primary or metastatic samples were received from the Cooperative Human Tissues Network and the Seattle Cancer Care Alliance. Tumor samples were processed as previously described to generate organoids. Human organoids were cultured with 2% (v/v) or 100% growth factor reduced Matrigel (Corning) with HuMEC Ready media (Fisher 12752010).

Scoring Metastatic Foci and Outgrowth after Tail Vein Injection

Three weeks after tail vein injection, lungs were removed and imaged under a dissecting microscope. Bright field and DsRed images were taken for all experiments using mTomato-PyMT cells, GFP images were collected for cells expressing GFP-shRNAs. To measure outgrowth in whole lungs or lung sections, fluorescence images were thresholded equally in FIJI and total fluorescent area was measured. For each tail vein injected mouse, the total area of lung metastases was measured as the cm² GFP+ area in the lungs using FIJI software. For each animal, the total area of lung metastases was normalized by the mean total metastatic area of Ctrl-kd mice within each cohort of tail vein injections. This allowed the effect size of control vs. gene knockdowns to be compared, accounting for intrinsic differences in organoid line growth rates between injections. Statistical analysis was performed using one-way ANOVA followed by Dunnett's Test for multiple comparisons. To measure individual metastasis areas, the area of each visible metastasis was measured manually using FIJI software. To identify and measure micrometastases, 50 urn thick lung sections were imaged at 10× magnification, tiled, and assembled. Metastasis counts from lung sections were normalized to the area of the lung imaged.

Immunofluorescence

Cells were fixed with 4% paraformaldehyde (10 min), permeabilized 30 minutes with 0.5% Triton-X, and blocked 1 hr at room temperature with 10% FBS/1% BSA/0.1% Triton-X in DPBS. For cells in suspension culture or in malignant ascites, cells were centrifuged onto tissue pathology slides using a Cytospin 4 (A78300003), 800 rpm for 5 minutes. Slides were fixed for 10 minutes in 4% paraformaldehyde and then treated as above. Primary antibodies were added in block solution and incubated at 4° C. overnight. Secondary antibodies (1:200) were incubated for 2-3 hrs at room temperature with 5% host serum. Confocal mages were acquired using an Andor CSU-W confocal spinning disk on a Leica DMi8 inverted microscope. For Leica 3×STED imaging, cells were stained with Alexa Fluor 594 Phalloidin (ThermoFisher Scientific A12381) and imaged using a Leica TCS SP8. DAPI was not included for STED imaging due to high background.

Time-Lapse Imaging for Apoptosis, Migration, and Outgrowth Analysis

Single or clustered cells were plated in growth factor reduced basement membrane-rich gels. Differential interference contrast (DIC) and fluorescent images were captured hourly using a Leica SPE at 10× or 20× magnification. For growth assays, DIC images were acquired. For survival assays, 30 minutes before imaging NucView 488 (Biotium) in PBS was added to the media at 1:1000 to mark nuclei in cells with active executioner caspases. Exposure times were <30 ms for BF/DIC, and ˜150 ms for fluorescence. Temperature was maintained at 37° C. and CO₂ at 5%. Motility and NucView positivity were used to score apoptosis in single cells and clusters. Clusters were marked as dead when the large majority of cells had died i.e. if a few cells were still alive in the apoptotic debris after most the cluster died, the cluster was not scored as alive. FIJI software was used to score the area of cells or clusters to measure outgrowth (as final area/initial area). Objects smaller than 250 μm² were scored as single cells (this cutoff may have occasionally included small MMTV-PyMT doublets). Images were acquired using a Leica DMi8 TCS SPE. For migration analysis, the centroid position of each individual organoid was tracked over time relative to static objects in the gel (e.g. debris). Each migration track was then normalized by the number of cells per organoid, determined by dividing the area of the organoid at time=0 hr by the mean μm² area per cell, which here it was determined to be 76 μm² (n=71 organoids, 8613 cells). Cumulative path was determined by summing over the path length of each migration track.

Mixed Culture Experiments Measuring Epgn-Kd Outgrowth with Non-Transduced Neighbors

On day zero, 22,500 cells were plated in non-adherent 96 well plate at 100% knockdown, 90% knockdown/10% non-transduced (e.g. 20250 knockdown cells, 2250 non-transduced cells), 50% knockdown/50% non-transduced, or 10% knockdown/90% non-transduced. On day 6, each well was centrifuged and resuspended in Accumax for 20 minutes at 37° C. Dissociated single cells were resuspended in organoid media and plated in glass-bottomed 8-well plates, allowed to settle, then imaged at 40× magnification (at least 10 fields per well). GFP positive and negative cells were manually counted in each field. For GFP+ cell growth calculations, this measurement was normalized to the starting number of GFP+ knockdown cells plated.

Transmission Electron Microscopy (TEM)

Tumor cell clusters from cell lines, organoids, patient samples, or primary tumors (minced with a scalpel to <1 mm³) were centrifuged, washed in DPBS, then fixed in ½ strength Karnovsky's buffer (2% PFA/2.5% glutaraldehyde in 0.1M cacodylate buffer) at 4° C. at 16 hrs. Samples were then processed by the Fred Hutchinson Cellular Imaging core. Samples were visualized using a JEOL-1400 transmission electron microscope operated at 120 kV. For ascites-derived samples, tumor cells were identified based on gross morphological differences from stromal and blood cells in the same sample, as well as by the presence of electron dense cell-cell junctions.

Immunoelectron Microscopy

For pEGFR and epigen immunogold staining, MMTV-PyMT clusters were collected and fixed in 4% PFA. For HER2 immunogold staining, MMTV-PyMT clusters were collected in 4% PFA+0.1% glutaraldehyde. Approximately 70 nm ultrathin sections were picked up on nickel grids (from Ted Pella, Inc., performed by Fred Hutchinson Cellular Imaging core). Grids were blocked with 50 mM glycine for 20 minutes, washed in PBS 3 times, blocked in 5% BSA for 30 minutes, washed with incubation buffer (1% BSA-C, 0.16% Tween-20 in PBS) 6×4 minutes, incubated with primary antibody (R&D MAB11271, Abcam ab40815, or CST 2165) for 2 hrs in incubation buffer, washed in PBS 4 times, washed in incubation buffer 6×4 minutes, incubated with secondary antibody (10 nm gold goat-anti-rabbit or goat-anti-rat), washed in incubation buffer 10×4 minutes, post-fixed in 2% glutaraldehyde for 10 minutes, and washed in warm DI water. Samples were visualized using a JEOL-1400 transmission electron microscope with a Gatan Rio 4K camera. Images were compared against a negative control not incubated with primary antibody to ensure secondary specificity.

Biotin Permeability Testing and Immunofluorescence

To test the permeability of tumor cell clusters, NHS-biotin was added to a final concentration of 0.8 mM in PBS for 30 minutes at 37° C. Treatment with 1 mM EGTA, Latrunculin A, or Ochratoxin A was for 1 hr, prior to biotin incubation. Treatment with interferon gamma was for 6 days prior to biotin permeability experiments. Cells were washed three times with cold PBS then centrifuged onto tissue pathology slides using a Cytospin 4 (800 rpm for 5 minutes) and fixed with 4% PFA for 10 minutes. Freshly dissected primary tumors or lungs were immersed in 0.8 mM biotin in PBS for 30 minutes then washed 3 times with PBS prior to fixation and embedding in OCT. Immunofluorescence was conducted (as above) with FITC-conjugated streptavidin to localize biotin. Human breast cancer cell lines were cultured for 6 days as clusters in suspension before assessing biotin permeability.

Immunofluorescence Quantification

Phalloidin staining and DAPI were used to define membrane and nuclear areas, respectively. Using the freehand line tool in FIJI software, lines along cell-matrix (outer membrane), cell-cell (intercellular) membranes, and intracellular (cytosolic) areas were drawn and median fluorescence intensity was measured for each channel. Values were normalized to cytosolic signal. For biotin leak-in assays, the freehand line tool was used to draw along the cell-matrix membranes of the cluster and measure FITC-streptavidin intensity. Then the polygon selection tool was used to measure FITC-streptavidin intensity in the cluster core (excluding the cell-matrix contacts) and intensity was normalized to the area measured. To assess biotin permeability in tumor vs. stroma areas, mean FITC-streptavidin signal was measured in FIJI in equally sized squares in adjacent pure tumor or pure stroma areas. To score protrusive vs. non-protrusive epigen immunofluorescence intensity, the polygon tool in FIJI software was used to measure epigen signal in locally disseminated mTomato+ PyMT clusters. Only protrusive and non-protrusive clusters in the same field of view were compared to one another, to account for differences in staining intensity between different regions of the tumor and different biological replicates. To count DAPI+ nuclei in clusters, a single confocal slice from the thickest part of the cluster was used. To assess cell death in clusters, propidium iodide (a fluorescent dye excluded from viable cells) was added to cell media at 0.5 ug/mL for 10 minutes prior to imaging.

Lentivirus Production and Transduction

Lentivirus was produced in HEK293FT cells using PsPax and MD2.G packaging plasmids. Supernatants were concentrated using Lenti-X (Clontech), resuspended in 1/100^{th} the supernatant volume of PBS, and frozen at −80° C. Viruses were titered using a p24 ELISA (Retrotek, calculated as 10 TU per picogram p24). Tumor organoids were dissociated to single cells in Accumax and resuspended at 150,000 viable cells/mL in organoid media+2% (v/v) matrigel. Protamine sulfate was added at 8 ug/mL to enhance transduction efficiency. For plasmids with PuroR, 2 days after transduction puromycin was included in the media at 1-2 ug/mL for selection. Cells were maintained in puromycin for at least 5 days before experiments and puromycin was included in the media throughout ex vivo culture. CRISPR sgRNA transduced cells were kept in puromycin at least 10 days before conducting experiments.

RTK Array and Quantification

The R&D Proteome Profiler Mouse Phospho-RTK Array Kit (R&D ARY014) was performed according to manufacturer instructions using 125 ug of dissociated single cell or clustered (6 hrs aggregation) MMTV-PyMT lysates. X-ray film was exposed to membranes for 1 to 10 minutes. To quantify signal, the pixel intensity of each coordinate was measured using FIJI software for film scans in which the signal was not over or under exposed. The signal of the negative control was then subtracted from each measurement.

Western Blotting

Clusters were lysed in RIPA buffer with protease and phosphatase inhibitors for 30 minutes at 4° C. and then centrifuged to remove debris (15 minutes at 12000 g). Clusters were cultured in 2% basement membrane suspension prior to lysate collection. Protein concentration was quantified using a BCA assay (Pierce), lysates were loaded onto a 4-12% Bis-Tris NuPage protein gel. Semi-dry transfers (iBlot2, P3 5-8 min) were performed using PVDF membranes which were then blocked with 5% BSA in TBST for one hr. Primary antibodies were incubated overnight at 4° C. in 1% BSA in TBST diluted 1:1000. Species specific LI-COR 680 and 800 secondary antibodies were used in TBST plus 1% BSA. Primary antibodies used: ERK1/2 (CST 4695), pERK1/2 (CST 4370), pAkt (CST 4060), Akt (CST 4691), beta-tubulin (Abcam ab40815), EGFR (Millipore 06-847). Band intensity was measured using the “Analyze Gels” tool in FIJI software.

Mammary Fat Pad Orthotopic Transplantation

MMTV-PyMT-mTomato organoids were resuspended in 50% basement membrane-rich gel in DMEM/F12 (vol/vol) on ice. 3-4-week-old NSG mice were anesthetized with 2.5% isoflurane and the surgical site was sterilized with ethanol and chlorhexidine. A 1 cm midline incision was made, allowing the #4 mammary fat pad to be exposed. 50,000 clustered cells per gland (aggregated at 250,000 viable cells/mL overnight) were injected into the left and right #4 mammary gland. The surgical area was locally infiltrated with 0.25% bupivacaine for pain relief. Surgical wounds were closed with 9 mm autoclips and tissue glue. Triple antibiotic ointment was applied to the incision. Mice were monitored closely with autoclip removal two weeks after surgery. Tumor volume was measured based on caliper measurements as: (4/3)×(p)×(width/2)×(width/2)×(length/2). At 10 weeks, mice were euthanized, and primary tumors and lungs were fixed in 4% PFA for 4 hrs, then transferred to 25% sucrose in DPBS overnight at 4° C. before embedding in OCT and storing at −80° C. Lung metastases were quantified using a fluorescence dissecting microscope at endpoint.

RNA Sequencing and Bioinformatic Analysis

MMTV-PyMT cells or clusters in suspension culture (non-adherent plates in media+2% Corning matrigel) were collected for RNA extraction. Cells were pelleted 5 min at 300 g then resuspended in 1 mL of Trizol. Samples were incubated at room temperature for 5 min. to ensure lysis, and then stored at −20° C. RNA samples were quantified using Qubit 2.0 Fluorometer and RNA integrity was checked with Agilent TapeStation (Agilent, RIN range 8.7 to 9.3). RNA sequencing libraries were prepared by GENEWIZ using the NEBNext Ultra RNA Library Prep Kit for Illumina following manufacturer's instructions. The sequencing libraries were validated on the Agilent TapeStation and quantified by using Qubit 2.0 Fluorometer as well as by quantitative PCR (KAPA Biosystems, Wilmington, Mass., USA). The sequencing libraries were clustered on 1 lane of a flowcell. After clustering, the flowcell was loaded on the Illumina HiSeq instrument (4000 or equivalent) according to manufacturer's instructions. The samples were sequenced using a 2×150 bp Paired End (PE) configuration. Image analysis and base calling were conducted by the HiSeq Control Software (HCS).

Raw sequence data (.bcl files) generated from Illumina HiSeq was converted into fastq files and de-multiplexed using Illumina's bcl2fastq 2.17 software. One mismatch was allowed for index sequence identification. Raw sequencing data was demultiplexed to generated two fastq files per sample with between 24.1 to 32.7 million reads per sample. Kallisto pseudoalignment (v0.46.0) was used for transcript abundance estimation, and differential transcript expression analysis was performed using Sleuth (v0.30.0). Kallisto was run with bootstrap-samples=100, and the transcript target was Ensembl (release 97) Mus musculus transcriptome (Mus_musculus.GRCm38.cdna.all.fa). For gene-level analyses, aggregated transcripts per million (TPM) scaled using the average transcript length and averaged over samples and to library size were generated with tximport and were used in further downstream differential gene expression analysis using limma-voom.

To visualize expression dynamics, genes were sequentially ordered in the dataset by their time-point of maximum expression, mean-variance normalized, and clustered together by time-point of maximum expression. Gene cluster enriched biological processes and signaling pathways were identified using Metascape.

RNA Sequencing Data Sets

Human RNA sequencing data from the Genotype-Tissue Expression (GTEx) Project were accessed using the GTEx portal (https://www.gtexportal.org/home/gene/EPGN). Breast cancer cell line RNA sequencing data sets were accessed from the Broad Institute CCLE portal (hops://portals.broadinstitute.org/ccle) or EMBL-EBI (hops://www.ebi.ac.uk/arrayexpress/experiments/E-MTAB-2706/).

Epigen ELISA

Epigen ELISAs were performed according to manufacturer instructions (R&D DuoSet) using MMTV-PyMT single cell or day 1 cluster (18 hrs aggregation) lysates in RIPA buffer diluted at least 5-fold in ELISA reagent diluent. At least 30 ug of total protein was loaded. Recombinant epigen (R&D 1127-EP) was used as a standard. Antibodies MAB11271 (R&D) and BAF1127 (R&D) were used for coating and detection, respectively.

Real-Time qPCR

Human or mouse tumor cell clusters were pelleted and snap frozen in liquid nitrogen. RNA was extracted using an RNEasy mini kit (Qiagen). 250 ng-1 ug of RNA was used to generate cDNA with a SuperScript III First-Strand Synthesis kit (ThermoFisher) with oligo(dT) primers. RT-qPCR was performed using PowerUp SYBR Green master mix in 10-20 uL reactions on a QuantStudio 5 real-time PCR instrument. Data were analyzed using the ΔΔCt method.

Quantification and Statistical Analysis

Bars are presented as mean±standard deviation. Red lines denote medians, unless otherwise noted. Graphs were created and statistical tests conducted in GraphPad Prism 8. Non-parametric tests were used when data were not normally distributed or when the median was a better representation of the sample than the mean. Experiments using cell lines on different days or using organoids generated from different mice were considered biological replicates. All statistical tests are two-sided. p<0.05 was considered significant. P-values: “ns” p>0.05, *p≤0.05, **p≤0.01, ***p≤0.001, ****p≤0.0001.

1. A method of inhibiting growth or metastasis of a solid tumor, comprising:
reducing the concentration of functional epigen in a microlumenal space between two or more cells of the solid tumor.

2. The method of claim 1, wherein reducing the concentration of functional epigen comprises reducing expression of epigen in one or more solid tumor cancer cells defining the boundary of the microlumenal space.

3. The method of claim 2, wherein reducing the expression of epigen in the solid tumor cancer cells comprises inducing RNA interference of the translation of epigen.

4. The method of claim 1, wherein reducing the concentration of functional epigen comprises administering to the microlumenal space an affinity reagent that selectively binds epigen.

5. The method of claim 1, wherein reducing the concentration of functional epigen comprises administering to the microlumenal space an affinity reagent that competes with epigen for binding to a cell surface receptor.

6. The method of claim 4, wherein the affinity reagent is an anti-epigen antibody, or antibody fragment or derivative.

7. The method of claim 1, wherein reducing the concentration of functional epigen comprises administering an effective amount of an agent that disrupts cell to cell contact of two or more cells defining the boundary of the microlumenal space.

8. The method of claim 7, wherein the agent disrupts calcium-dependent cell to cell contact.

9. The method of claim 8, wherein the agent is a calcium chelator, such as ethylene glycol-bis(β-aminoethyl ether)-N,N,N′,N′-tetraacetic acid (EGTA), ethylenediaminetetraacetic acid (EDTA).

10. The method of claim 1, wherein the solid tumor is a breast cancer tumor, or a tumor derived therefrom.

11. A method of treating a cancer in a subject in need thereof, wherein the cancer is characterized by a solid tumor, a circulating tumor cell (CTC) cluster, and/or a disseminated tumor cell (DTC) cluster, comprising:
administering to the subject an effective amount of an agent that reduces the functional concentration of epigen in an extracellular microlumenal space between two or more cells of the solid tumor, CTC cluster, or DTC cluster.

12. The method of claim 11, wherein reducing the concentration of functional epigen comprises administering an effective amount of an agent that disrupts cell to cell contact of two or more cells defining the boundary of the microlumenal space.

13. The method of claim 12, wherein the agent disrupts calcium-dependent cell to cell contacts.

14. The method of claim 13, wherein the agent is a calcium chelator, such as ethylene glycol-bis(β-aminoethyl ether)-N,N,N′,N′-tetraacetic acid (EGTA).

15. The method of claim 11, further comprising administering to the subject an effective amount of an immunotherapeutic agent for the cancer.

16. The method of claim 15, wherein the immunotherapeutic agent is a checkpoint inhibitor.

17. The method of claim 15, wherein the immunotherapeutic agent comprises a cancer-specific antibody or functional fragment thereof.

18. The method of claim 15, wherein the immunotherapeutic agent comprises an immune cell modified or expanded ex vivo and which expresses a receptor specific for the cancer.

19. A method of determining whether tumor cells are cluster-dependent, comprising:
obtaining a cluster of tumor cells; and
detecting spatial distribution of a low-affinity EGFR ligand within the cluster of tumor cells;
wherein localization of the low-affinity EGFR ligand within a microlumenal space between two or more tumor cells of the cluster indicates the cluster dependency of the tumor cells.

20-29. (canceled)

21. A method of treating breast cancer in a subject in need thereof, comprising administering to the subject an effective amount of interferon gamma (IFNγ).

22-36. (canceled)

23. The method of claim 1, further comprising contacting the solid tumor with an effective amount of interferon gamma (IFNγ).

24. The method of claim 11, further comprising administering to the subject an effective amount of interferon gamma (IFNγ).
